(19) United States
(12) Patent Application Publication
(10) Pub. No.: US 2023/0242972 A1
(43) Pub. Date: Aug. 3, 2023

(54) SINGLE-BEAD CAPACITIVE DETECTOR FOR MICROBIOLOGICAL APPLICATIONS

(71) Applicant: Altratech Limited, Limerick (IE)

(72) Inventors: Tara Dalton, Limerick (IE); Timothy Cummins, Cratloe (IE); Margaret Aherne, Limerick (IE); David McGuire, Limerick (IE); John O'Driscoll, Fountainstown (IE); Colin King, Limerick (IE); Paul Free, Haverhill (GB); Brian O'Farrell, Watergrasshill (IE)

(21) Appl. No.: 18/102,355

(22) Filed: Jan. 27, 2023

Related U.S. Application Data
(60) Provisional application No. 63/304,312, filed on Jan. 28, 2022.

(51) Int. Cl. C12Q 1/6837; G01N 27/22; G01N 33/543; G01N 33/53

(52) U.S. Cl. CPC C12Q 1/6837; G01N 27/226; G01N 33/54313; G01N 33/5308; C12Q 1/6804

(57) ABSTRACT

The present invention provides an improved capacitive bead sensor for detection and/or quantification of target analytes in a sample, with a detection limit down to single-beads, which is re-usable for multiple bead tests, or for a continuous flow of beads, and which is easily manufacturable and automatable. It enables sensitivity down to single molecule detection without the need for enzymatic amplification such as PCR, by use of various structural advantages and electronic signal amplification techniques that further allow for multiplex target detection not only across various nucleic acid targets but across entire target classes allowing for simultaneous detection of viral nucleic acids and host antibodies to that virus for example.

### RELATED APPLICATIONS

The present application claims the benefit of and priority to U.S. provisional patent application Ser. No. 63/304,312, filed Jan. 28, 2022, the content of which is incorporated by reference herein in its entirety

### FIELD OF THE INVENTION

The present invention relates generally to methods of nucleic acid and antibody/antigen molecular capture by magnetic beads, and detection/quantification of these molecules, to the level of single-bead and consequently single-molecule detection.

### BACKGROUND

Nucleic Acid (NA) capture and detection from biological samples has become a critical aspect of early disease detection and monitoring. In a conventional lab clinical assessment, both molecular and serological (antibody/antigen) testing is used together in providing a valid diagnosis. Ideally, such tests would be done together from the one sample. As an example, a PCR or LAMP test can provide qualitative or perhaps semi-quantitative information about the presence of a viral RNA while separate antibody detection can establish a person's previous infection status. Quantification of captured NA is important, for example, in determining viral-load of an infectious pathogen, or for gene-expression analysis of multiple captured NAs. Multiplex testing, variant identification, and genotyping has also become very important, as seen in the recent COVID19 pandemic, where ‘variants of concern’ (e.g. Delta, Omicron) caused huge health issues and many deaths. Direct single molecule detection with ultra-high sensitivity may allow analysis of rare mutations which might cause cancer, detection of trace contamination of biopharma products with pathogenic DNA or RNA, very early or late detection of viral infection in patients where viral load is quite low (see ‘single-molecule-PCR’ U.S. Pat. No. 5,811,235, incorporated herein by reference), and for cloning and template-generation in DNA and RNA sequencing. A drawback of ultra-sensitive methods can be cross-contamination with amplicons, incidental contact of a suspect with a crime scene and patients continuing to test positive for the presence of pathogenic RNA or DNA long after they have recovered from the infection and are no longer infectious. These are a particular feature of enzymatic amplification methods.

These critical health diagnostics are currently available only in highly specialized laboratories, require highly trained staff and significant equipment and infrastructure all contributing to significant expense.

The use of magnetic beads for nucleic acid capture is known. Optimal magnetic bead diameters for this range from 400 nm to 2800 nm. Non-magnetic particles (for example, glass, silica, polystyrene, titania, silver, or gold) are also employed for nucleic acid capture, with even smaller diameters, down to 40 nm typically. A notable use of nanoparticles in diagnostics is in lateral flow tests where they are conjugated with antibodies for the colorimetric detection of antigen or, vice versa, conjugated with antigen for antibody testing using the same technology.

Captured DNA is eluted, and if the target DNA is present and hybridizes with matching oligomer probes and primers, it is typically amplified exponentially in a Polymerase Chain-Reaction (PCR) or Loop-mediated isothermal amplification (LAMP) assay for optical detection. A reverse-transcription step (RNA to cDNA) is first employed if the captured target is RNA.

Conventional enzymatic amplification is employed for detection of the target NA if present. However, precise quantification of target NA in the sample is difficult with these PCR and LAMP methods due to the stochastic nature of exponential amplification, and samples with low concentrations are still difficult to measure and quantitate. Single-molecule detection using PCR and LAMP is also difficult due to enzymatic inhibition with particular samples, and the length of the primer probes, which are typically >18 (commonly 20+) and >50 nucleotides respectively. This can prevent binding to critical mutations such as those in the receptor binding domain (RBD) of the spike gene of SARS-CoV-2 virus, where many of the single-base-mutations (e.g. in Delta or Omicron variants) can occur within 15 to 20 nucleotides of each other. Droplet/digital-PCR and sequencing can address single-molecule detection and discrimination, but those methods typically require large and quite expensive laboratory instruments and trained specialist operators.

Detection of a magnetic bead carrying a target molecule has been proposed on a complementary metal-oxide semiconductor (CMOS) chip, for example the Hall-sensor, as described in Florescu et al. (2010) “On-chip magnetic separation of superparamagnetic beads for integrated molecular analysis” J Appl. Phys. 107(5):054702, and the GMR-SV-sensor, as described in Murmann et al. (2013) “A 256 pixel magnetoresistive biosensor microarray in 0.18 μm CMOS”, IEEE J Solid-State Circuits 48(5):1290-1301, the content of each of which is incorporated by reference in its entirety herein.

However, each of these CMOS chips require extra magnetic and metallic special layers, which are expensive and not generally available on standard high-volume CMOS foundry processes.

Chang and Lu (2013) “CMOS capacitive biosensors for highly sensitive biosensing applications”, Annual Int Conf IEEE Eng Med Biol Soc. 2013:4102-5, the content of which is incorporated herein by reference in its entirety, proposes capacitive detection of a magnetic bead on a CMOS chip.

Detection of non-magnetic particles such glass, silica, titania, silver, gold nanoparticles typically requires expensive optical detectors, lasers, X-ray or SEM, or mass-spectrometry instruments. Visual detection with the human eye is possible in some Lateral-flow tests, in which large numbers of typically 40 nm nanoparticles are captured at the detection (or control lines) enable visual detection. But sensitivity of these methods is limited to thousands of particles.

U.S. Pat. No. 10,746,683 (Cummins et al), the content of which is incorporated herein by reference in its entirety, describes an interdigitated electrode (IDE) capacitive sensor for detecting particles, both magnetic and non-magnetic as listed above. This is a single-use sensor, with a lower detection limit of 200 beads, in which the water or buffer carrier evaporates, leaving the beads permanently attached to the sensor surface.

U.S. Pat. No. 10,160,966 (O'Farrell et al), the content of which is incorporated herein by reference in its entirety, discloses beads which may be provided to a sample, and peptide-nucleic-acid (PNA) probes attached to these beads which hybridize and capture target nucleic-acid molecules. This is a single-use arrangement.

U.S. Pat. No. 11,459,601 (O'Farrell et al), the content of which is incorporated herein by reference in its entirety, discloses various assay steps for nucleic acid detection. Paramagnetic transport (T) beads, with first PNA probes and captured nucleic-acid attached, are magnetically removed from the sample and moved through various wash and tether steps. Reporter (R) beads with second PNA probes attached then tether to the captured target nucleic acid, if present, creating a target-specific sandwich assay. This is then moved to a wash chamber and onto a CMOS sensor chip, where the R-beads are eluted and detected. This is also a single-use arrangement.

### SUMMARY OF THE INVENTION

Systems and methods of the present disclosure generally relate to detection and/or quantification of target analytes in a sample. Through the various structures and techniques discussed herein, alone or in combination, sensitivity down to single molecule detection can be obtained without the need for enzymatic amplification such as PCR. Systems and methods of the invention can use various structural advantages and electronic signal amplification techniques that further allow for multiplex target detection not only across various nucleic acid targets but across entire target classes allowing for simultaneous detection of viral nucleic acids and host antibodies to that virus for example.

Aspects of the invention can include methods for nucleic acid detection including detecting a target nucleic acid present in a sample at 100 copies/mL or less using a capacitive sensor and without enzymatic amplification. In various embodiments, the techniques discussed throughout can be used alone or combined to achieve detection of 150 copies/mL or less, 250 copies/mL or less, 500 copies/mL or less, 1000 copies/mL or less, 5000 copies/mL or less, 10,000 copies/mL or less, 50,000 copies of mL or less, 100,000 copies of mL or less, 1,000,000 copies/mL or less and so on depending on the desired sensitivity, accuracy, and complexity of the assay. In certain embodiments, methods may be operable to achieve at least 10⁹ signal amplification in detection of the target nucleic acid. In various embodiments, methods may achieve at least 10⁸, at least 10⁷, at least 10⁶, at least 10⁵, at least 10⁴, at least 10³, or at least 10², signal amplification, again depending on the assay parameters. Similarly, methods may be operable to achieve signal amplification in detection of the target nucleic acid equivalent to at least 30 PCR cycles, at least 25 PCR cycles, at least 20 PCR cycles, at least 15 PCR cycles, at least 10 PCR cycles, or at least 5 PCR cycles.

Nucleic acid detection by capacitive sensor can include a full-scale range of at least about 8 pF, at least about 7 pF, at least about 6 pF, at least about 5 pF, at least about 4 pF, at least about 3 pF, at least about 2 pF, or at least about 1 pF. Methods may include converting a signal from the capacitive sensor using a capacitive-to-digital converter. The capacitive-to-digital converter may be a sigma-delta 24-bit capacitive-to-digital converter. Nucleic acid detection by capacitive sensor can include at least about 0.5 aF resolution and at least about 4 aF accuracy. In various embodiments, nucleic acid detection by capacitive sensor may include a full-scale range of at least about 15 pF through inclusion of internal offset capacitors in a reference input.

Methods of the invention may further include binding the target nucleic acid to a reporter (R) bead; passing the R-bead through a sensor region comprising two capacitive electrodes on a substrate in communication with a signal processing circuit; and detecting the R-bead as it passes through the gap using the signal processing circuit. The two electrodes may be spaced apart to form a gap such that only a single bead moves between the gap at a time. The two electrodes may form a trench through which the R-bead passes. Methods may further comprise passing the R-bead through a plurality of sensor regions; and detecting the R-bead as it passes through the plurality of sensor regions by applying one or more of a Maximum Likelihood Estimation (MLE) machine-learning algorithm, a Partial-Response Maximum Likelihood (PRML) algorithm, or a Viterbi algorithm to signals received by the signal processing circuit from the plurality of sensor regions.

In some embodiments, detecting the target nucleic acid can include binding the target nucleic acid to a reporter (R) bead, and detecting the R-bead using a capacitive sensor. Detecting the R-bead using the capacitive sensor can include detecting the R-bead flowing past the sensor in a fluid. The fluid may be an oil. The oil can be a silicone oil. The fluid may comprise fluorinated carbons. The fluid can be selected from Dodecafluoro-2-methylpentan-3-one and methoxy-nonafluorobutane. Methods may include fluorinating the R-bead. Methods may comprise forming an aqueous layer around the R-bead flowing in the fluid to amplify a detection signal at the capacitive sensor.

In certain embodiments, methods may include binding the target nucleic acid to the R-bead using a bead-bound peptide nucleic acid (PNA). The bead-bound PNA can include a ligand or linker PNA which doesn't interact with target RNA. Methods may comprise detecting a plurality of different targets comprising at least the target nucleic acid present in a sample using one or more capacitive sensors. The plurality of different targets can include a plurality of different target nucleic acids. The plurality of different target nucleic acids may be derived from different pathogens or different variants of a pathogen. The pathogen may be SARS-CoV-2. The plurality of different targets can comprise a protein. The protein may be an antibody. The protein may be an antigen. In some embodiments, the target nucleic acid may be derived from Dengue virus and the antibody can be a Dengue-specific antibody. In certain embodiments, the target may be a drug, including but not limited to Onpattro, Patisiran, givosiran, lumasiran, and inclisiran (siRNAs) or antibody cocktails such as REGEN-COV (casirivimab and imdevimab). The target could be a mRNA vaccine such as tozinameran. Co-monitoring of therapeutics and viral load is advantageous and the multiplex nature of the present methods and systems can allow for such co-monitoring. Stability of some RNA-derived drugs is a concern both before use and after use and a monitoring technology for the concentrations or levels of these both prior to injection, say, and from a blood or saliva sample after use is of value. The same technology could be used for QC of these technologies in pharma companies. The target could include target nucleic acid derivatives which may be synthetic and include non-standard bases such as N1-Methylpseudouridine. The target could also be a phospholipid or glycolipid. Targets could be on the surface of a cell, viral particle or lipid nanoparticle wherein the cell, viral particle or lipid nanoparticle allows tethering.

Detecting the plurality of different targets may include binding each of the plurality of targets to a different bead; and detecting each of the different beads using the capacitive sensor. Methods may further comprise differentiating the different beads based on different capacitive detection signals from the capacitive sensor. In some embodiments, methods may include selectively releasing the R-beads from the substrate-bound targets based on the different sensor-bound target to which they are bound; and detecting the effect on capacitance of releasing the R-beads.

Detecting the plurality of different targets can include binding each of the plurality of different targets to a different probe on a substrate. The substrate may be operably associated with one or more capacitive sensors. Methods may further comprise binding one or more reporter (R) beads to each of the different substrate-bound targets. In some embodiments, methods may include selectively releasing the R-beads from the substrate-bound targets based on the different substrate-bound target to which they are bound; flowing the released R-beads past the capacitive sensor in a fluid; and detecting the released R-beads flowing past the sensor. Each of the different beads may have a different size and methods may include microfluidically directing each of the different beads to a different capacitive sensor based on its size. The directing step can comprise one or more of inertial, dielectrophoretic or magnetophoretic methods.

Detecting the target nucleic acid can further comprise binding the target nucleic acid to a probe on a substrate; and binding a plurality of reporter (R) beads to the substrate-bound target nucleic acid. The plurality of R beads may bind to different sequences in the substrate-bound target nucleic acid. The substrate can be operably associated with the capacitive sensor. Methods may further comprise releasing the plurality of R-beads from the substrate-bound target nucleic acid; flowing the released R-beads past the capacitive sensor in a fluid; and detecting the released R-beads flowing past the sensor.

In certain embodiments, detecting the target nucleic acid can include binding a transport (T) bead to the target nucleic acid in the sample to form a T-bead complex; binding the T-bead complex to a reporter (R) bead to form an R-bead complex; eluting the R-bead and the T-bead from the R-bead complex; detecting the R-bead using a capacitive sensor; and returning the T-bead to the sample to bind another target nucleic acid.

Aspects of the invention can also include systems and architecture as described herein operable to perform any of the methods described above.

Aspects of the invention may include a system for nucleic acid detection, the system comprising a capacitive sensor further comprising a signal processing circuit and a sensor region comprising two capacitive electrodes on a substrate in communication with the signal processing circuit wherein the two electrodes are spaced apart to form a gap and wherein only a single bead moves between the gap at any time and the electrodes detect the bead and/or any molecules attached to the beads. The system may be reusable after being used to detect beads. The capacitive sensor can be approximately 2 mm×2 mm. The bead or particle may be contained in water, buffer, or oil or fluorinated carbon, or organic solvent. The bead or particle being detected may be a proxy for a microbiological analyte from an upstream assay. The bead or particle may be magnetic or non-magnetic.

The gap may be 2× the bead diameter. In some embodiments, the substrate may be 3D printed. The electrodes can be formed by sputtering or ink-deposition and patterning. The spacing between electrodes may be about 0.8 μm to 20 μm. The substrate can be a CMOS semiconductor chip. The electrodes may be etched in a metal layer and the spacing between electrodes can be between about 40 nm and about 5 μm. In certain embodiments, systems and methods of the invention may further comprise one or more of ultrasonic shaking, dielectrophoretic bead-steering, oil syringing an/or tuning of the bead zeta potential to prevent bead agglomeration and/or induce movement of particles and beads through the system.

It is an object of this invention to make critical health diagnostics available outside of a laboratory, in a simple portable format, requiring little or no training for operation, for use at point-of-care, and in the home. However, the same technology may reduce the size, scale, power consumption and logistical costs of central laboratories. It is a further object of the invention to enable simultaneous and rapid detection of nucleic-acid and antibodies from a patient sample. This simultaneous detection can be advantageous for cases such as a Dengue outbreak, where it is vital not just to detect and distinguish between Dengue virus (DENV) RNA variants 1,2,3,4 (indicating a current active infection), but to also detect and identify DENV antibodies. In triaging, it can be helpful to establish whether the patient had a previous historical Dengue infection, before making any clinical decisions about applying Dengue vaccine.

Systems and methods described herein recognize and address certain shortcomings in the techniques discussed above. For example, Florescu's bead is quite large, typically 2.8 μm or bigger, and requires precise manipulation of the bead onto a circular Hall-Sensor of 4.5 μm diameter. This fine manipulation of single particles is not practical in an automated commercial NA assay. Murmann's magnetic particles are smaller (50 nm), but the lower limit of detection is 2000 particles, rendering it impractical for most Nucleic Acid tests and gene-expression analysis. The bead described in Chang and Lu is 10 μm diameter, too large for most molecular analysis assays, and requires electromagnetic manipulation of the bead into the precise center of a coil. This also makes it impractical for a commercial or automated assay. Moreover, the sensing mechanism described in Chang and Lu is not capacitive since Chang and Lu's capacitance actually reduces in the presence of a bead. Accordingly, there is an unmet need for a re-useable sensor which can detect a continuous flow of beads and detect single beads. There is further an unmet need for a continuous flow of beads to enable single-molecule analysis. There is also an unmet need for a continuous flow of T-beads and R-beads to enable single-molecule analysis, genotyping, and variant identification as well as an unmet need for a simple portable instrument and method to perform nucleic acid detection, variant identification, viral load quantification, and single-molecule detection analysis, in a non-laboratory setting, such as point-of-care and community clinics, to allow rapid clinical intervention, treatment, and real-time monitoring in epidemic outbreak situations.

### BRIEF DESCRIPTION OF THE DRAWINGS

FIG. 1 depicts analyte droplets with beads on sputtered ITO electrodes on PET film.

FIG. 2 depicts capacitive to digital converters and a connection socket to the PET film.

FIG. 3 depicts a 24-bit sigma-delta capacitive-to-digital converter architecture and circuit.

FIG. 4 depicts ink-printed laser-ablated electrode capacitors on a 3D-printed substrate.

FIG. 5 is a side-view photo of an ink-printed laser-ablated electrode of FIG. 4.

FIG. 6 depicts an etched aluminum electrode IDE capacitor on a CMOS silicon chip.

FIG. 7 depicts aluminum and copper electrode CMOS semiconductor formation.

FIG. 8 depicts a prior art analyte sensor with tethered bead, and its equivalent circuit.

FIG. 9 is a photo of beads specifically captured on the circular CMOS sensor of FIG. 6.

FIG. 10 depicts end-to-end assay performance in a graph of Capacitance-vs-RNA.

FIG. 11 depicts signal amplification by specific tethering of many beads to one sensor.

FIG. 12 depicts a PNA probe targeting a conserved region of the SARS-CoV-2 virus.

FIG. 13 & FIG. 14 depict further PNA probes targeting regions of SARS-CoV-2 virus.

FIG. 15 depicts antigenic peptide sequences of Dengue Envelope and NS1 proteins.

FIG. 16 and FIG. 17 depict PNA probes targeting Dengue virus variants 1,2,3,4.

FIG. 18 depicts thirty of the CMOS sensors with PNA probes spotted on some sensors.

FIG. 19 depicts a spotting pattern of the sensors for Dengue RNA and antibody detection.

FIG. 20 depicts Dengue RNA & Antibody assays side-by-side on the CMOS sensor chip.

FIG. 21 depicts several of the IDE capacitors of FIG. 6 in rows on a CMOS silicon chip.

FIG. 22 is a photo of the chip of FIG. 21 with transparent silicone oil on some sensors

FIG. 23 is a schematic of a cylinder of the oil over one of the FIG. 21 circular sensors.

FIG. 24 depicts the equivalent circuit of FIG. 23 with C_{silicone-oil} and C_{nitride}.

FIG. 25 is a photo of a PD5 oil-film containing beads on a circular sensor of FIG. 21.

FIG. 26 is a schematic of the cylinder of oil with beads of FIG. 25 on the circular sensor.

FIG. 27 depicts the equivalent circuit of FIG. 26 with C_{silicone-oil}, C_{nitride}, C_{bead}.

FIG. 28 provides sensor measurements of PD5 oil capacitances with and without beads.

FIG. 29 depicts a 0.4 μm bead in a 0.8 μm trench between metal electrodes.

FIG. 30 is an electric field simulation of this bead in oil passing between the electrodes.

FIG. 31 depicts the calculated capacitance of the bead as it passes between the electrodes.

FIG. 32 depicts the boundary-element-model used in the simulations of FIGS. 30 & 31.

FIG. 33 depicts a ground-plane just beneath the electrodes of FIG. 29.

FIG. 34 shows the bead capacitance calculation of FIG. 33, with ground-plane beneath.

FIG. 35 shows the real-time capacitances of a series of beads flowing between electrodes.

FIG. 36 depicts beads flowing between multiple electrodes, then down a hole in substrate.

FIG. 37 depicts a laser-formed through-hole in ink-electrodes and a 3D-printed substrate.

FIG. 38 depicts ink-printed electrodes with a narrow gap formed by laser ablation.

FIG. 39 depicts an assay according to certain embodiments with recycling of T-beads.

FIG. 40 depicts an embodiment to enable single-molecule analysis, e.g. of SARS-CoV-2.

FIG. 41 depicts a home respiratory saliva self-test embodiment for COVID/Flu/Hay-fever

FIG. 42 illustrates fluorinated reporter beads flowing through a fluidic phase transition.

FIG. 43 illustrates a microfluidic embodiment for flowing a bead across phase transitions.

FIG. 44 depicts R-beads tethered to D-beads which provide additional amplification.

FIG. 45 shows the R-bead-D-bead tethered complexes flowing between sensors.

FIGS. 46A-46B show the R-D bead complex tethered to a CMOS sensor in FIG. 46A and a bead substrate in FIG. 46B.

FIG. 47 shows a reduced size R nanoparticle (1-100 nm) tethering a D bead to surface.

FIG. 48 shows the R nanoparticle having a linker-type molecule shape.

FIG. 49 shows the linker being a ligand which does not interact with target RNA.

FIG. 50 shows the R nanoparticle tethering a D-bead to surface of a magnetic particle.

### DETAILED DESCRIPTION

The present invention provides an improved capacitive bead sensor which is re-usable for multiple bead tests, or for a continuous flow of beads, and which is easily manufacturable and automatable. Systems and methods described herein allow for detection down to single-beads and, therefore, single target molecules. Through electronic and other signal amplification techniques discussed below, that sensitivity can be accomplished without enzymatic amplification, thereby avoiding some of the drawbacks of those enzymatic methods as described above. Furthermore, systems and methods described herein can achieve the desired sensitivity while maintaining a wide dynamic range particularly useful in multiplex analysis of target molecules at varying concentrations in a sample.

As discussed in more detail below, the systems and methods described herein can allow for, among other things, one or more of a) capture and detection of nucleic acid, antibody, and antigen molecules simultaneously from a biological sample; b) quantification of these molecules (e.g. for clinical viral-load determination) down to single-digit copy level; c) gene-expression analysis by simultaneous quantification of multiple PNA-captured genes on multiple capacitive bead sensors; d) single-molecule detection analysis by multiple PNA-probes on multiple beads recycled through the sample and the assay steps; and e) discrimination of current infection from past infection by simultaneous detection of both RNA and antibodies from the same sample.

### Bead Detection and Signal Amplification

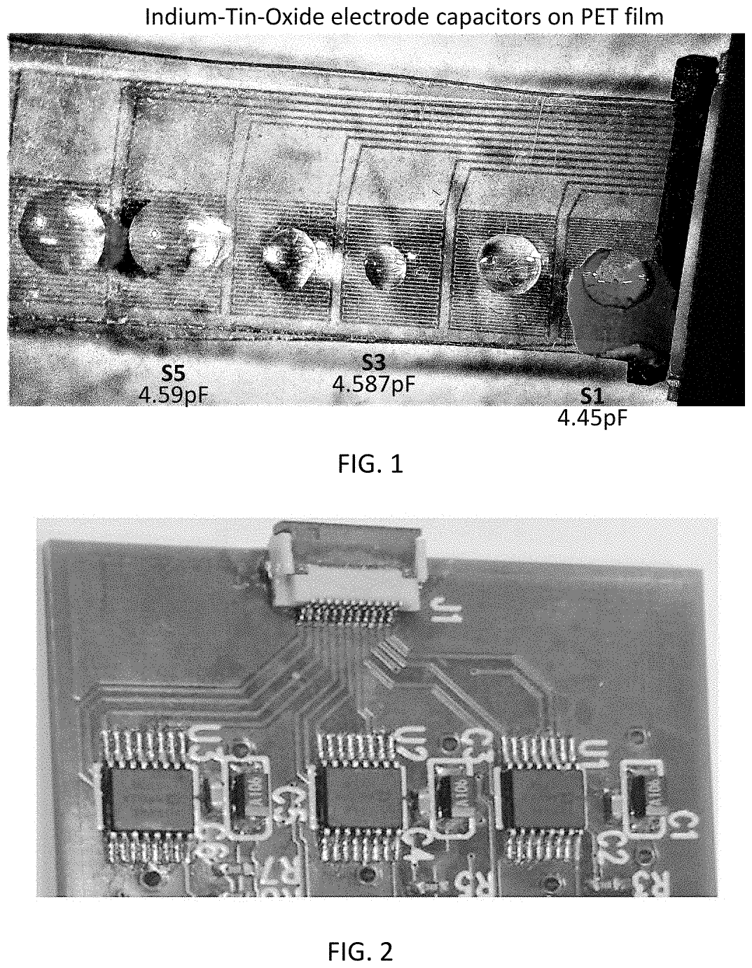

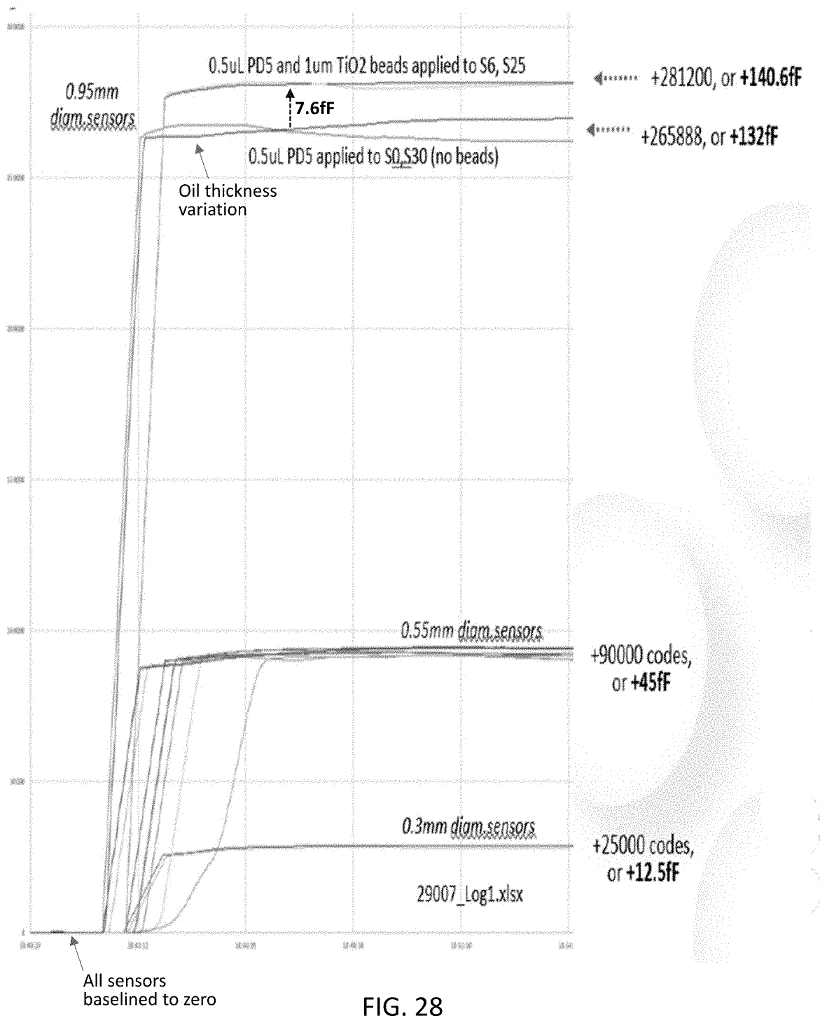

FIG. 1 depicts six sputtered Indium-Tin-Oxide (ITO) capacitive electrode sensors on a polyethylene terephthalate (PET) film of 150 μm thickness. Each capacitor is approximately 2 mm×2 mm. A connector socket (at right) connects to capacitors to a signal processor, which may include capacitance-to-digital converters, analogue and digital filters, non-volatile memory, a signal processor, and wireless communication (Bluetooth, WiFi, GSM, GPRS, etc). The capacitances shown (4.45 pF, 4.587 pF, 4.59 pF) are baseline values without liquid or beads. The slight value increases (S1-S3-S5) reflect the longer wires to reach each capacitor. These baseline values can be stored as offsets in the non-volatile memory of the signal-processing circuit, to enable nulling of all sensors to a zero baseline by offset subtraction from each of the relevant sensor readings (as shown in FIG. 28). The sensors are arranged in a linear row, to facilitate integration with droplet dispensing tips, for assay automation in some applications.

FIG. 2 depicts capacitance-to-digital converters on a printed circuit board (PCB) and a miniature socket connecting these to the six capacitors of FIG. 1.

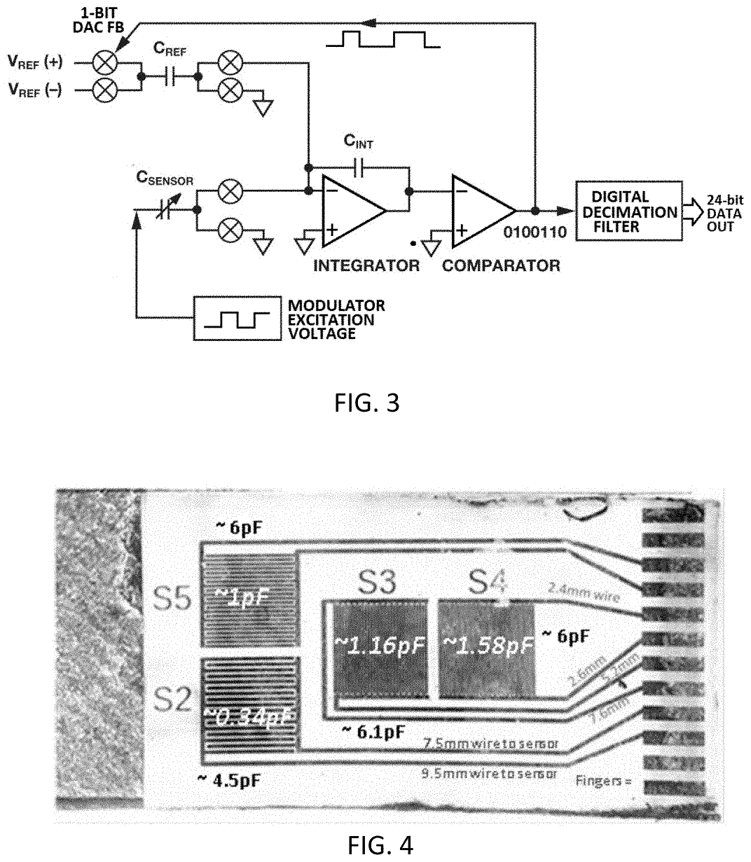

FIG. 3 depicts the circuit architecture of a sigma-delta capacitive-to-digital converter, as is known in the art, e.g. “Oversampling Delta-Sigma Data Converters” (Candy et al, Wiley-IEEE Press, 1992). Using standard switched-capacitor techniques, operating at a modulator clock frequency of KHz to MHz, Cₛₑₙₛₒᵣ is compared to C_{ref}; the difference is amplified and integrated, then applied to a comparator. The comparator output is fed back to control the V_{ref} switches, effectively a 1-bit digital-to-analog convertor (DAC). The comparator bit-stream output is applied to a decimation filter to produce a digital result, of up to 24 bits resolution. Typical conversion times are 1 mS to 100 mS. The million-fold electronic amplification provided by the multi-stage integration amplifier can contribute to the overall assay amplification.

FIG. 4 depicts a 3D-printed polylactic acid (PLA) substrate with interdigitated electrodes formed using ink-printing and laser-ablation patterning. The measured baseline capacitances (including fingers and wiring parasitics) are in the region of 4.5 pF to 6.1 pF. The fringe-field sensing or transduction portion of capacitance may be in the region 0.4 pF to 1.6 pF, depending on electrode spacings, which may typically be from 0.8 μm to 20 μm, depending on the spot-size of the ablation laser.

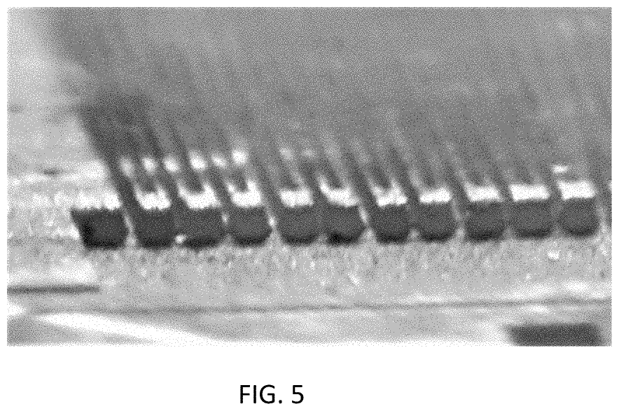

FIG. 5 is a side-view photograph of the ink electrodes of S4 in FIG. 4. Laser ablation of the ink to create the electrode structures results in trenches between the electrodes, with height of the ink-thickness, which is programmable from a few microns to hundreds of microns during 3D printing. Electro-Hydro-Dynamic ink-jet printing is another method of creating fine-line (e.g., 1 μm) electrode features without need of laser-ablation.

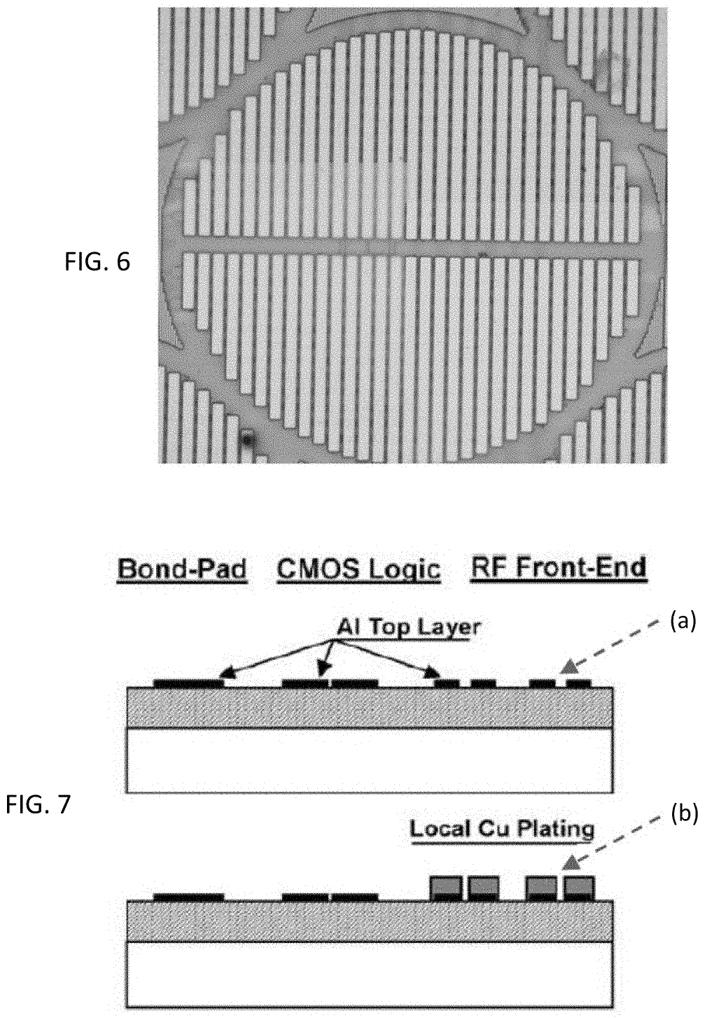

FIG. 6 depicts an interdigitated electrode (IDE) capacitor formed by subtractive etching of the top metal layer of a Complementary Metal-Oxide Semiconductor (CMOS) chip. In this embodiment the sigma-delta capacitive-to-digital converter may be formed in the same CMOS chip, underneath or adjacent to the sensors. This significantly reduces parasitic capacitances and noise (thermal and electromagnetic), thereby giving a wider dynamic range of measurement.

A cross-section view of the metal layers which form the CMOS sensor is shown in FIG. 7 (an illustrative example from Burghartz et al, IEEE 2004, DOI 10.1109/TED.2004.823325). This reference also describes typical CMOS process formation steps and is incorporated herein by reference in its entirety. Electrode spacings as low as 20 nm can be achieved with modern dry-plasma-etch and highly directional anisotropic deep reactive ion-etch (DRIE) methods of semiconductor fabrication. The dashed line (a) in FIG. 7 shows the narrow-spaces in top-metal which may be used as trenches for flowing beads in-between, without passivation, in certain embodiments. This can be advantageous in eliminating a manufacturing step (passivation) to achieve narrower spacings while providing increased assay amplification. Oxidation or corrosion of the metal does not occur when covered in silicone oil in which the beads may flow. The dashed line (b) in FIG. 7 is a variation with copper plating for much thicker metal structures, similar to the 3D-printed trenches of FIG. 5. This copper-plating example in Burghartz is for RF inductor coil formation. In certain embodiments, systems of the invention may use this standard high-volume RF-CMOS thick metal technique as electrode trench structures, for measurement of beads in the trenches, and the measurement of the flow of beads in oil. This can avoid the requirement for special CMOS layers or non-standard semiconductor processing.

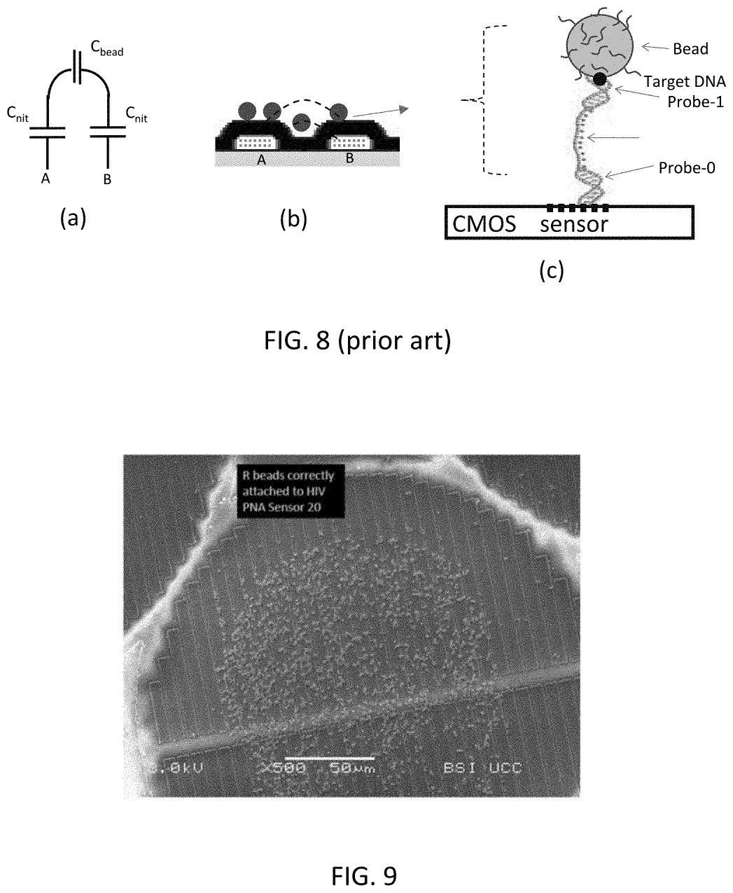

FIG. 8 shows a capacitive bead detection method (see U.S. Pat. No. 10,746,683, incorporated herein by reference in its entirety). Beads may simply arrive on the sensor surface (8b) or be specifically tethered to the sensor surface (8c), for example by Probe-0, and tethered by a further Probe-1 attached to the bead. The capacitive fringe-field (8b dotted lines of electric field) forms the transducer, detecting C_{bead} due to dielectric constant of the bead material. 8(a) shows the equivalent circuit. C_{nitride} appears in series with C_{bead}, since the field lines pass through the insulating nitride protective layer.

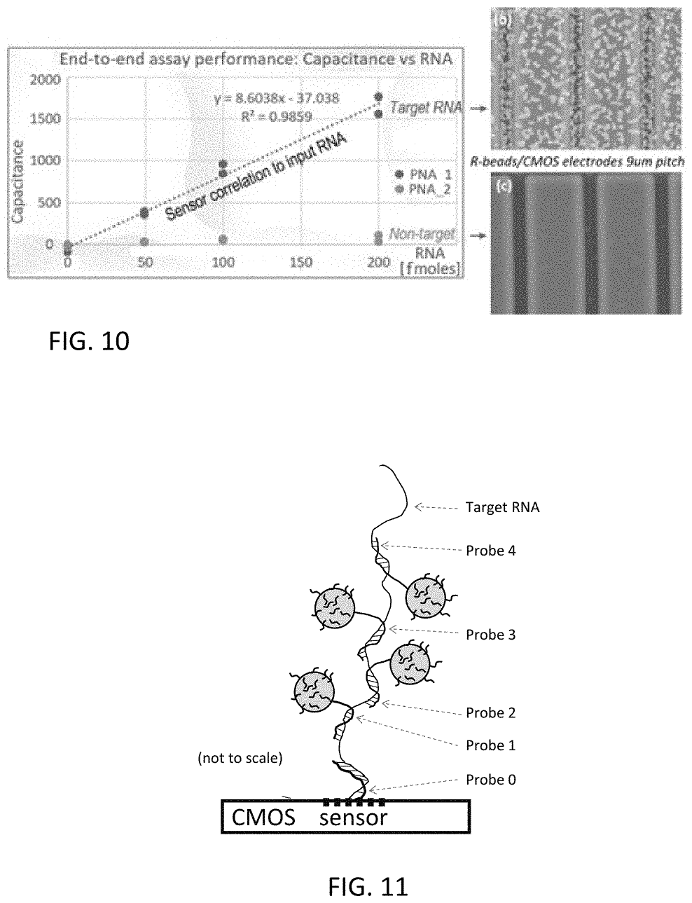

FIG. 9 is a photographic illustration of FIG. 8(c) tethering, in this case showing reporter beads (R beads) specifically captured on the center of the circular sensor of FIG. 6 by PNA probes designed to target the HIV gag gene. Since these are proxy beads for the HIV-RNA in the upstream assay from the patient sample, the sensor capacitance, being a quantification of the number of beads, is also a direct digital readout of the HIV viral-load in the patient's sample. This is illustrated in the end-to-end assay performance graph of “Capacitance versus RNA” in FIG. 10. The top photo shows beads specifically captured on the sensor surface. The bottom photo shows no beads are captured when an off-target PNA probe is used.

FIG. 11 shows a target RNA strand tethered to the sensor surface (by the probe-0), with several beads tethered to the RNA, by probes 1,2,3,4 etc. These probes can be designed to target different conserved regions of a target DNA or RNA, thus giving very high specificity of target capture. It also results in significant signal-amplification, due to the increased number of captured beads tethered onto the sensor. This mimics dendritic amplification, and can aid overall assay amplification, for quantifying of DNA or RNA even at very low copy number levels without enzymatic amplification.

### Nucleic Acid and Antibody Assay Peptide and Probe Design

The sequences of the HIV Gag gene PNA probes of FIG. 9 have been published by Zhao et al. in Nature Communications (nComms 5079), the content of which is incorporated herein in its entirety. Fully synthetic PNA probes can be synthesised chemically by FMOC chemistry using solid-phase peptide synthesis (SPPS).

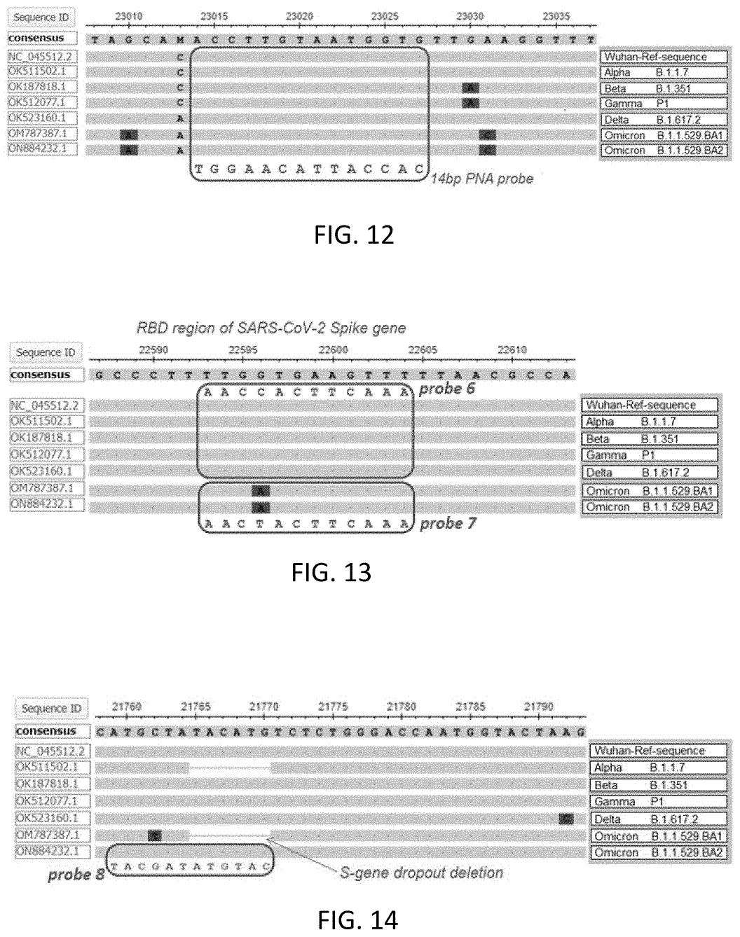

FIG. 12 shows a 14 base PNA probe targeting a unique region of the SARS-CoV-2 viral genome, conserved across the Wuhan reference sequence and Alpha, Beta, Gamma, Delta, and Omicron BA.1 and BA.2 variants. This probe may be tethered to the sensor surface, as probe-0 of FIG. 11 for example.

FIGS. 13 and 14 show further PNA probes (probe 6, probe 7) designed to target regions of the SARS-CoV-2 receptor binding domain (RBD) for discriminating the Omicron BA.1 and BA.2 variants from Alpha, Beta, Gamma, and Delta variants. Probe 8 further narrows and identifies BA.2 variant by targeting its S-gene dropout deletion. Further probes are similarly applied to identify other variants, as is known in the art, where variant identification and viral genotyping can be achieved with a small number of carefully designed probe sequences (e.g., by applying a logic table as shown in FIG. 41). E.g. in 2004 “A universal microarray for detection of SARS coronavirus” (doi: 10.1016/j.jviromet.2004.06.016), Long et al published 20 probe sequences giving “not only detection of SARS-CoV but also identification of the genotypes of six mutated bases related to the different phases of the SARS epidemic” (of 2003/2004).

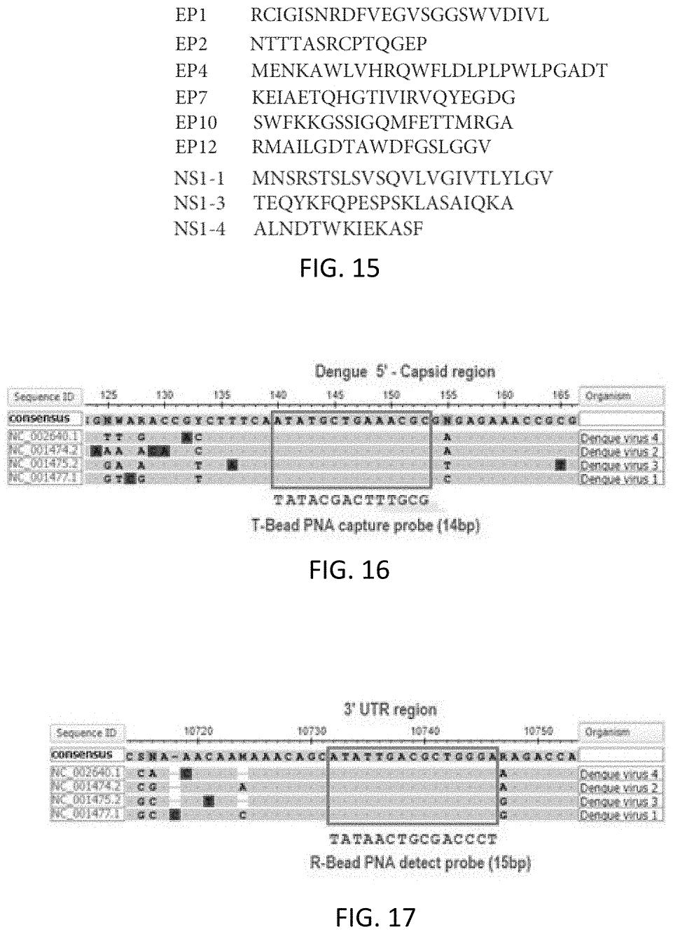

FIG. 15 shows synthetic peptide sequences of envelope (E) and non-structural (NS-1) proteins of the Dengue virus (DENV), (from Nagar et al, DOI 10.1155/2020/1820325). These may also be synthesized chemically by FMOC SPPS. They are based on linear epitopes of DENV envelope and non-structural proteins, used to test for and diagnose Dengue-specific IgM and IgG antibodies in the patient's antisera. The nine sequences shown are among the “most antigenic/reactive” for giving high-specificity antibody detection (from Nagar et al).

FIGS. 16 and 17 show an example of achieving 100% detection of the four Dengue reference genomes NC 001477, NC 001474, NC 001475, NC 002640 (DENV 1, 2, 3, 4 respectively), with just two PNA probes (14 bp & 15 bp) targeting the conserved regions shown. This is not possible with longer PCR probes and primers, which are >25 nt typically and would therefore span some of the mutations shown, resulting in less than 100% specificity, and also less capture efficiency. The high capture efficiency of PNA's can also contribute to the overall assay signal amplification and, therefore sensitivity.

### Multiplex Testing

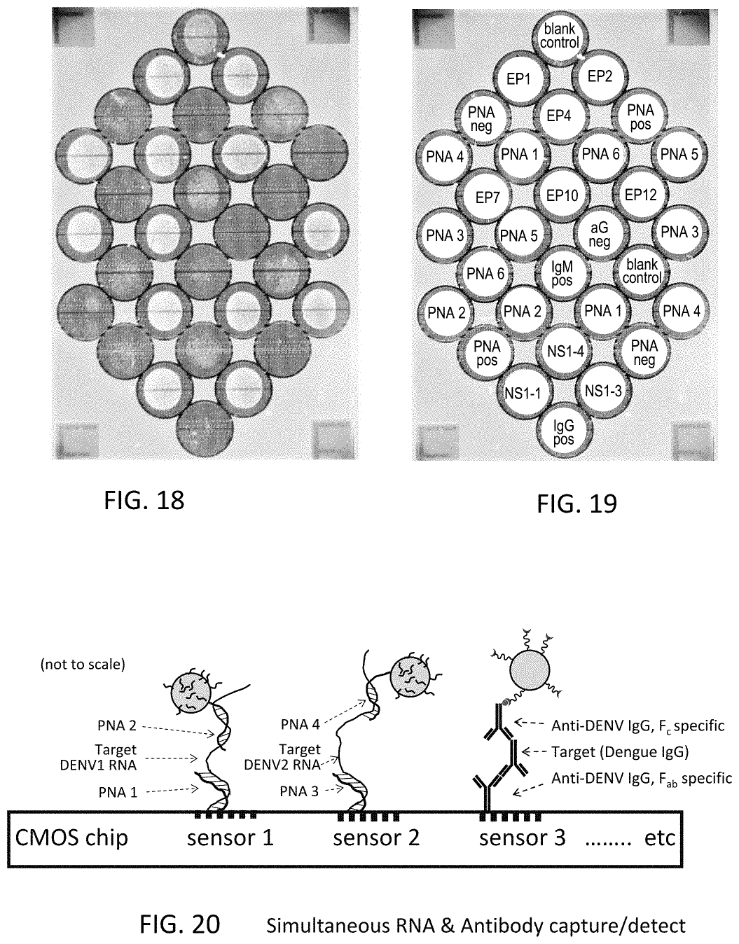

FIG. 18 is a photograph showing thirty circular CMOS sensors, as shown individually in FIG. 6, grouped together, with PNA tethering probes spotted onto various sensors. These probes can specifically capture reporter R beads, as seen in the bead-circle of FIG. 9 and illustrated in FIG. 11. The thirty sensors enable much higher-order multiplexing and genotyping than is possible with LAMP or PCR assays. This is illustrated in an exemplary embodiment in FIG. 19: Twelve sensors are spotted with dual-replicates of the six R-bead DENV PNA probes (PNA 1-6). These comprise two PNA probes of FIGS. 16/17 for 100% Dengue detection, and one each for DENV 1/2/3/4 mutation identification. Nine sensors are spotted with the E and NS1 peptide sequences of FIG. 15 for high-specificity DENV antibody detection: EP1,EP2,EP4,EP7,EP10,EP12,NS1-1,NS1-3,NS1-4. The bead-quantification ability of each of the sensors can be used to establish E and NS1 quantities. Combined with machine-learning capability of a CMOS chip electronics and processor, this can assist in enhancing assay sensitivity and selectivity, and in helping to predict ‘severe dengue’ and the potential onset of severe and potentially fatal Dengue Haemorrhagic Fever (DHF) and Dengue Shock Syndrome (DSS). Two PNA-positive control sensors (both spotted with 18s rRNA complementary probe if blood sample, or with RNAse P complementary probe if saliva sample) are included. Two ‘PNA-neg’ negative-control sensors, e.g. for Single-Base-Mismatch confirmatory test control are included. Sensors spotted for IgM and IgG positive controls, and aG negative antigen controls are also included. Two blank sensors, for temperature and CMOS process variation correction and offset eliminations are used.

This high-multiplex and nucleic-acid/antibody unique simultaneous detection capability of this assay is performed by the thirty co-located sensors, and the side-by-side, fully synthetic peptide sequences and peptide-nucleic-acid (PNA) probes, as shown in FIG. 20, each synthesized by the same FMOC chemistry.

In certain embodiments, the multiplexed detection described above may be performed using flow through detection. For example, each spot described above may not be a sensor. Instead, tethering of R beads to a substrate can be mediated by carefully designed PNA linker or ligand sequences which don't interact with RNA in the sample. These are conjugated to the PNA probes targeting the RNA or to an antigenic peptide sequence. Careful design of the linker PNA allows sequential and selective elution of R beads from the substrate as the system is heated and the melt temperature of the linker PNA is reached. In this fashion, R beads attached to spots mediated by RNA targets can be released selectively and sequentially followed by measurement on a downstream sensor. Further, R beads attached to spots mediated by antibodies can be released selectively and measured on a downstream sensor as the Tm of the relevant linker PNAs are reached. The sensor can be an inline sensor which detects the R beads flowing past them. In certain embodiments, the sensor can be a PNA functionalized sensor which binds the specific R bead. Alternatively, the substrate can be the surface of magnetic beads.

### Detection of Beads Flowing in a Silicone Oil

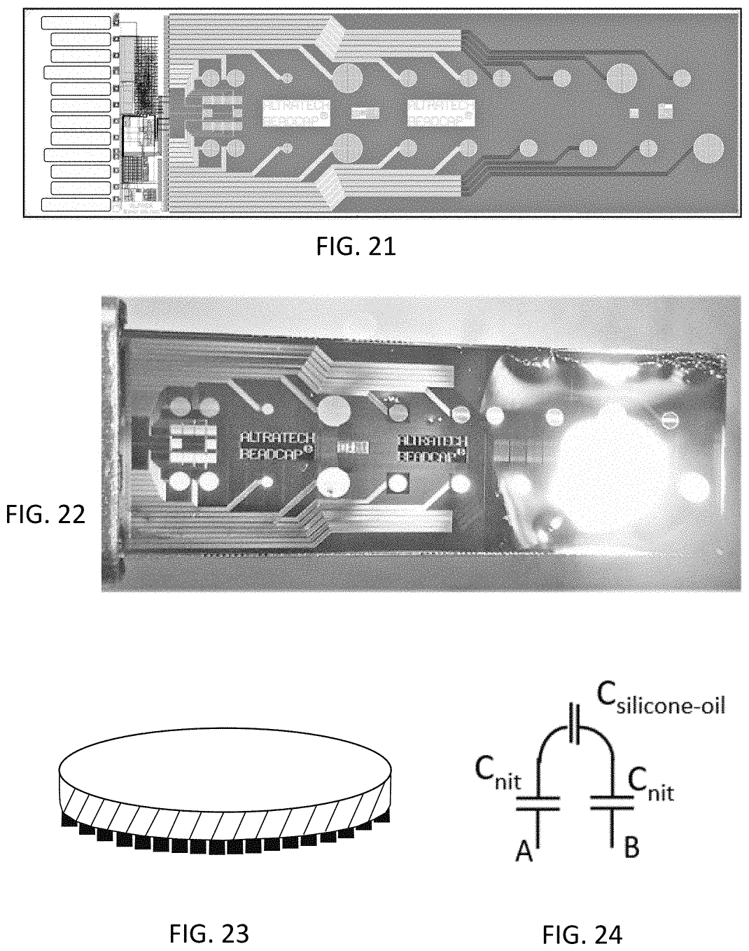

FIG. 21 depicts another embodiment of the sensing capacitors of FIG. 6 on a CMOS silicon chip. In this embodiment, the sigma-delta capacitance-to-digital converters are formed within the CMOS chip, at the left side of the sensing capacitors. Multiple diode temperature sensors integrated across the chip ensure that differences in sensor readings due to temperature variations across the chip can be measured and compensated for. The sensors are in a row and column offset formation, to facilitate integration with droplet dispensers for some assay automation applications, or multi-channel assay methods, e.g. where the upstream nucleic-acid and antibody assays may be in different channels.

FIG. 22 is a photograph of the chip of FIG. 21 with a film of transparent PD5 silicone oil on some sensors of various diameters on the right half of the chip. The film of oil sits in the fringe-field transduction portion of the IDE electrode capacitive sensors, and thus increases sensor capacitance due to the oil dielectric constant K of approximately 2.5 to 3, which is higher than air (K=1).

FIG. 23 is a schematic of a cylinder portion of the oil film being sensed over one of the circular sensors. FIG. 24 depicts the equivalent circuit of FIG. 23, showing the oil capacitance C_{silicone-oil} appearing in series with C_{nitride}, the capacitance of the insulating nitride passivation layer.

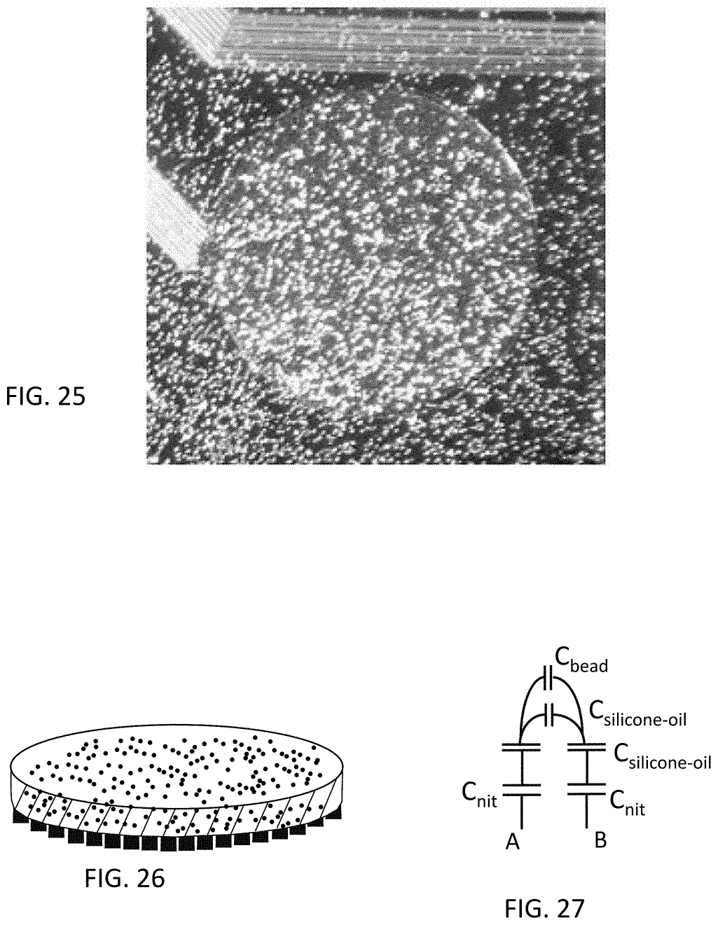

FIG. 25 is a photograph of a PD5 oil-film containing Titanium Dioxide (TiO₂) beads on one of the circular sensors. These Titanium Dioxide analyte beads in the oil film add further capacitance, due to their dielectric constant of ˜30 to ˜100, which is much higher than the oil dielectric constant (2.5 to 3). In fact, K of TiO₂ may be 10× higher (up to K=1000) at lower frequencies (100 Hz, as per Wypych et al, DOI 10.1155/2014/124814). Thus, a further 10× assay amplification is possible by slowing down the sigma-delta converter modulator frequency, at the cost of a slower conversion time of up to 1 second.

FIG. 26 is a schematic view of FIG. 25, showing a cylinder portion of the oil with beads on the circular sensor.

FIG. 27 is an equivalent circuit of this analyte sensor of FIG. 25 and FIG. 26, depicting the relevant C_{silicone-oil} and C_{bead} capacitances, in series with C_{nitride}.

FIG. 28 provides sensor measurements of oil capacitances with and without beads. At time zero, all sensors are baselined to zero with air-dielectric. After 2 minutes, a PD5 oil film (no beads) is applied to sensors S0 and S30 (as negative controls), and a minute later PD5 oil containing 1 μm TiO₂ beads is applied to S6 and S25. As can be seen at top of graph, the beads cause a 7.6 fF increase in capacitance. Visual counting of the beads on a microscope (as in FIG. 25 photograph) gives an estimate of approx. 6000 beads, i.e., indicating approx. 1.26 aF per bead. This equates to about 12 electrons, for a switched-capacitor voltage of 1.6V applied between the electrodes by the modulator of the sigma-delta converter.

Referring to S0 and S30 negative control sensors (0.96 mm diameter), the addition of the silicone oil at 2 minutes increases the capacitance by 132 fF. For the 0.54 mm and 0.3 mm diameter sensors S5 and S7, the capacitance increases by 45 fF and 12.5 fF respectively. These measurements are summarized in Table 1 below, which shows that the silicone-oil film is adding about 183 fF/sq·mm compared to air-dielectric, irrespective of sensor diameter:

TABLE 1

| | Diam | Area | Codes | | | |
|---|---|---|---|---|---|---|
| | (mm) | (sq · mm) | PD5 | fF | fF/sq · mm | |
| S0 | 0.96 | 0.72232 | 265888 | 132.9 | 184.1 | |
| S5 | 0.54 | 0.23758 | 90000 | 45.0 | 189.4 | |
| S7 | 0.3 | 0.07069 | 25000 | 12.5 | 176.8 | |
| | | | | | 183.4 | Avg |

These measurements of beads in oil illustrate various embodiments of systems and methods of the invention. They can be used to eliminate the need for liquid evaporation and are advantageous in allowing sensor re-use for multiple measurements. It can allow movement of beads by liquid flow control and syringing—useful for non-magnetic beads and particles to be detected, or for magnetic particles. The graph depicted in FIG. 28 also illustrates changes in capacitance when the oil-film thickness varies, and when beads flow away from the sensor, illustrating dynamic real-time bead-flow detection capability.

### Electrical Characterization of Single-Bead and Bead-Flow in Oil

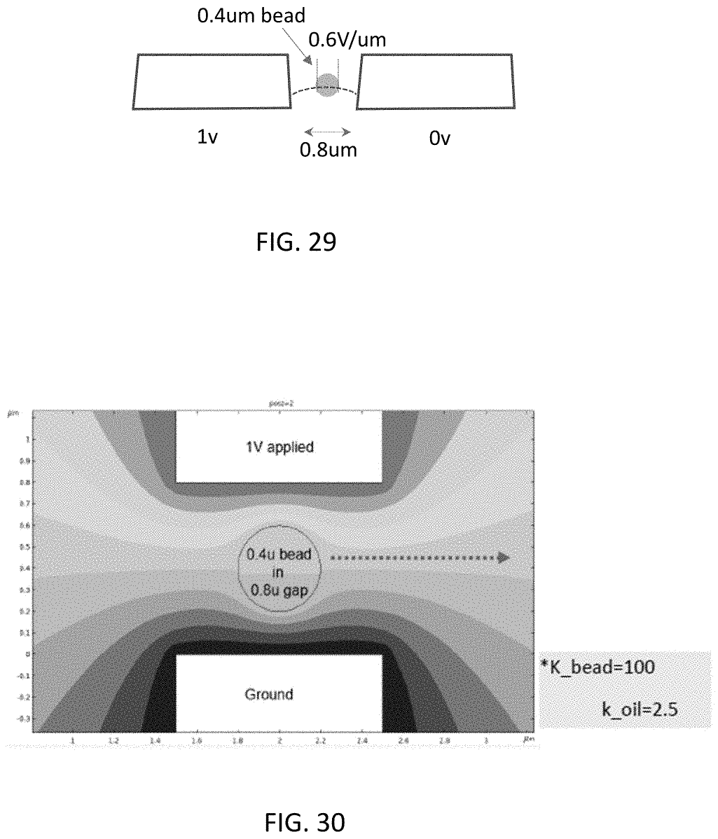

FIG. 29 depicts a 0.4 μm bead in the 0.8 μm trench between electrodes.

FIG. 30 shows a COMSOL electrostatic simulation of these electrodes with 1 volt applied, and of this bead (K=100), in oil (K=2.5), passing between the electrodes. The electric field gradient lines are shown in 0.1V steps.

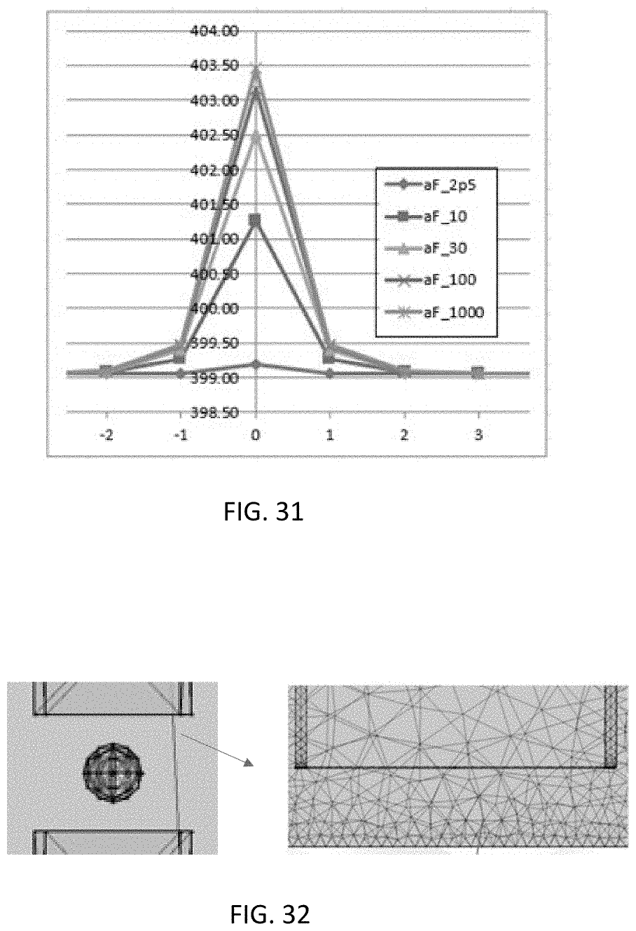

FIG. 31 shows the COMSOL capacitance calculations of the bead as it passes between the electrodes, for bead dielectric constants of 2.5, 10, 30, 100, and 1000. The capacitance peaks at 4 attoFarads (aF) as the 0.4 μm bead (K=100) in oil passes through the electrode gap of 0.8 μm. This is a higher capacitance for a 0.4 μm bead than the 1.2 aF for a bigger 1 μm TiO2 bead (from graph of FIG. 28), due to the smaller electrode spacing and stronger electric field across the bead. This can further contribute to the overall signal amplification of this non-enzymatic detection/quantification assay. Increasing the electric field across the bead increases dipole polarization of the molecules within the bead, thereby increasing its capacitance. The baseline capacitance of 399 aF is quite low in this simulation, due in part to short electrodes in the model with no other wires or ground planes in the vicinity, as shown in the FIG. 32 boundary-element-model (BEM) used in this simulation.

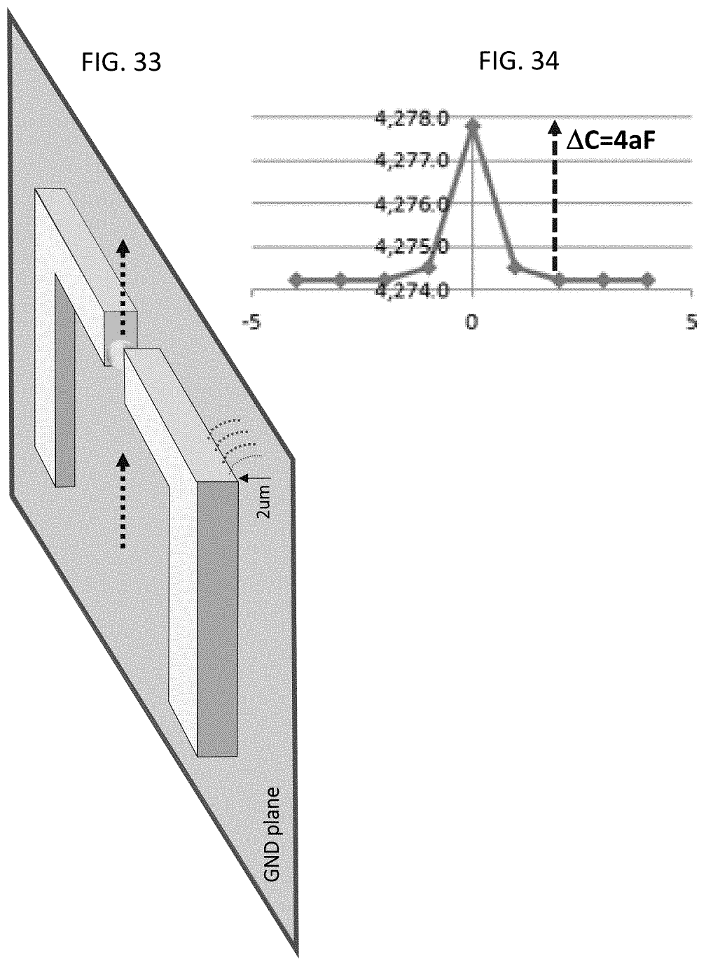

In practice the electrodes may have wires of a few millimeters in length connecting them to the capacitive-to-digital converter, and/or may be formed on a physical substrate, e.g. a 3D-printed base, or a CMOS silicon substrate. Electrically these substrates can appear as a large ground-plane. This is shown in FIG. 33, in which a 0.4 μm bead passes through a 0.8 μm electrode gap, with a ground-plane 2 μm underneath, as might be typical on a CMOS silicon substrate. For a K=100 bead in oil (K=2.5), the COMSOL bead capacitance is once again 4 aF as shown in FIG. 34. However the FIG. 34 baseline capacitance is 4.274 femtofarad (fF) (i.e. over 10× higher than in FIG. 31) due to the increased parasitic capacitance to the nearby ground plane. Longer wires (e.g., 10 mm to 15 mm) can increase this baseline further, for example by 4.5 pF to 6.1 pF in the embodiments shown in FIGS. 1 and 4. It is notable that the 4 aF single-bead capacitance is detectable even with these huge baseline variations from attoFarads (aF) to picoFarads (pF). This is due to the wide dynamic range of the sigma-delta 24-bit capacitive-to-digital converter. This embodiment has 8 pF full scale range, 0.5 aF resolution, and 4 aF accuracy. Extra internal offset capacitors can be added to C_{ref} (in FIG. 3), to extend the baseline range, for example up to 17 pF or 20 pF. This can allow manufacture of the capacitive sensor in many different ways, by 3D printing, with or without a ground plane, on a silicon substrate or CMOS chip even with large parasitics.

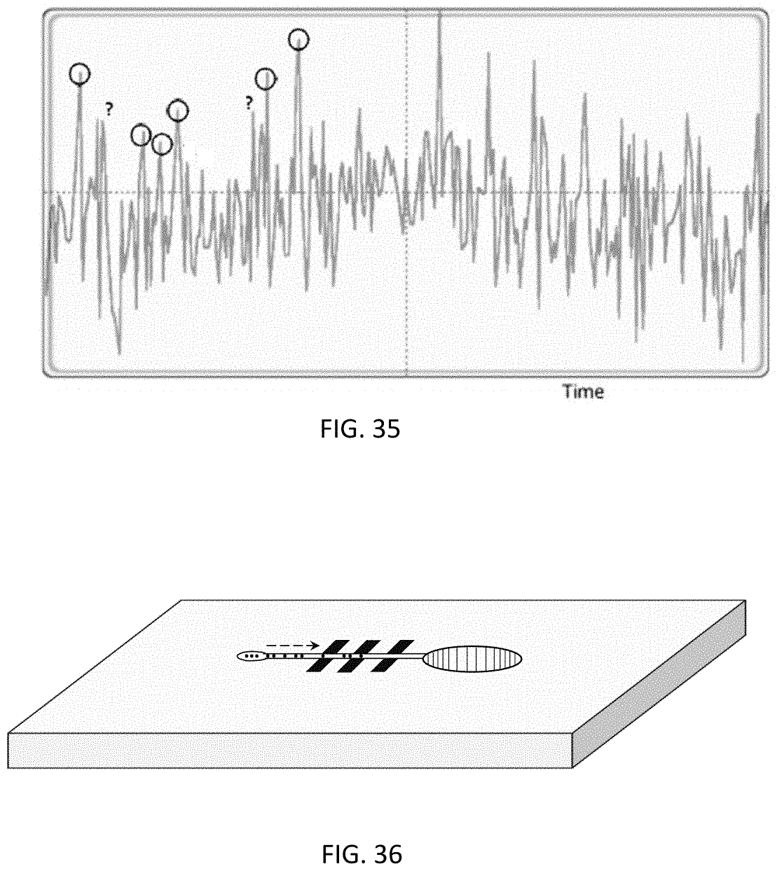

FIG. 35 shows a time-domain graph of converter output for very low attoFarad-level measurements. A large amount of thermal and 1/F noise is evident. Beads passing between electrodes may appear as a short ‘blip’ corresponding to 3 aF to 6 aF capacitive signal approx. (circled tips in waveform). Discriminating these blips is not straightforward. Simple peak-detection may miss ambiguous blips (exemplified by the peaks marked with “?” in FIG. 35). In certain embodiments, moving average filtering can be used to aid discrimination, but may miss short blips. Accordingly, in some embodiments, multiple electrodes may be used in the bead channel to help by reading the same bead multiple times as shown in FIG. 36. A Maximum Likelihood Estimation (MLE) machine-learning algorithm can then be applied to the readings to improve bead-count accuracy. Partial-Response Maximum Likelihood (PRML) and Viterbi algorithms further improve bead discrimination and reduce error rates. These digital-filtering improvements of signal-to-noise ratio, which may be embedded within the CMOS sensor chip itself, can further contribute to the overall assay amplification without enzymatic amplification.

Many amplification methods and steps have been described in this disclosure: PNA high capture efficiency, bead-mass, CMOS 10⁶ electronic amplification, multi-bead tethering signal amplification, bead electric field increase in trenches, MLE and PRML/Viterbi digital signal processing. The combined effect of these is to reach 10⁹ to 10¹² amplification in this assay. This is roughly equivalent to 30 to 40 PCR cycles, respectively. Thus the all-synthetic assays described herein can be used to achieve analytical sensitivity levels of <100 copies/mL, yet with a wide dynamic range of several orders, which is ideal for clinical RNA viral load monitoring. Assays of the invention can further eliminate the requirement for enzymatic amplification, thus also eliminating many of the inhibition issues and complexities of PCR and LAMP assays. Being all-synthetic, the assay also eliminates the requirement for dry-ice shipping and storage in refrigerators or freezers. Long shelf life, potentially up to years like an electronic device, is another possible benefit relative to enzymatic assays where reagents can deteriorate more rapidly and without special handling.

### Through-Hole Bead Flow Assay Methods

FIG. 36 depicts beads flowing along a channel or trench on the substrate, between multiple electrode pairs, then flowing down a hole in the substrate, exiting at the rear. In some embodiments, this may be a DRIE etched through-silicon hole, as described in WO2004/109770.

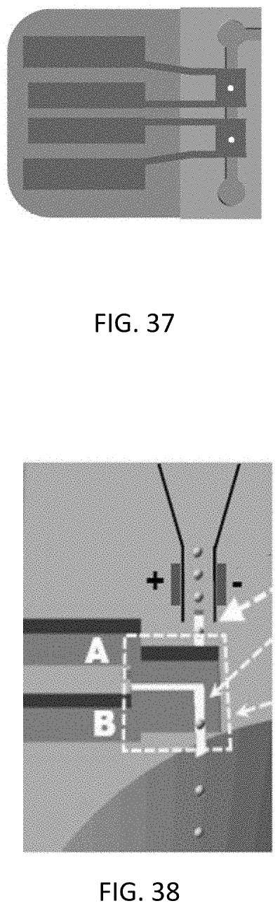

FIG. 37 shows an exemplary laser formed through-hole in the ink electrodes and a 3D printed PLA substrate. This can be advantageous for in-line bead measurements, where beads may be flowing in tubes or channels.

FIG. 38 depicts a narrow 0.8 μm electrode spacing between A and B ink-printed electrodes, formed by laser-ablation with a 0.8 μm laser spot size. Beads flow in a channel then ‘drop down’ through a hole between electrodes (created by laser ablation of the 3D-printed base). Smooth bead-flow can be enhanced by syringing push-pull control of the oil or other liquid flow carrying the beads, and/or by dielectrophoretic steering, in which the charged bead (e.g. −30 mV zeta potential) responds to positive or negative voltages applied from outside or along the flow channel, e.g. by the +/−tips shown in FIG. 38. Bead agglomeration can be minimized by tuning the bead Zeta potential, e.g. to −30 mV by carboxylic coating, which can cause the beads to repel each other, and/or by ultrasonic-shaking of the apparatus to aid in monodisperse flow through the sensor.

To provide multiplexing capability in a flow through detector system, multiple flow through sensors can be placed in the same device. Several discrete groups of R-beads of different sizes can be used corresponding to each variant to be tested. These R-beads may be spatially separated to allow each size group to be delivered to a different through-hole sensor. The method of separation can be through inertial separation, or dielectrophoretic separation or magnetophoretic separation. The beads can thereby be differentiated allowing for differential detection of each bead's respective target in a multiplex assay.

### Recycling of Beads

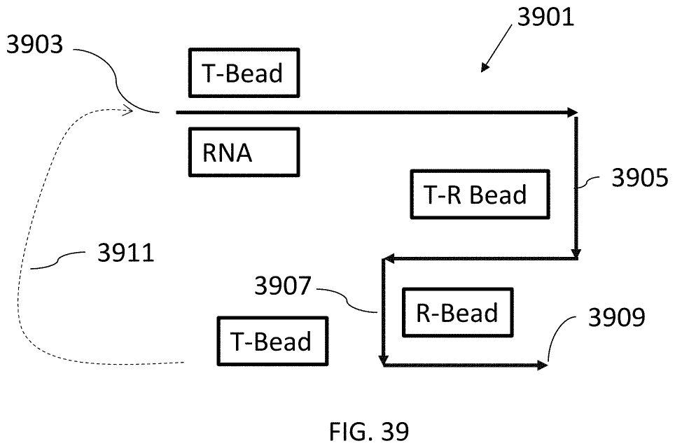

In certain embodiments, it may be advantageous to recycle the beads back through the system depending on the workflow. An exemplary bead recycling scheme is shown in FIG. 39. The system 3901 has four main areas, sample capture 3903, assay building 3905, elution 3907 and sensor detection 3909. Depending on the sensing method chosen, recycling the T-beads can have an advantage as there is a continuous supply of T-beads until the targeted RNA is depleted (FIG. 39). The T-bead captures the target RNA via a T-probe at 3903. The R probe and R-bead are added in the next stage of the assay building at 3905 at which point the full assay is transported magnetically to the elution point 3907 over the sensor. The eluted R-bead then attaches to the sensor 3909 via a specific PNA-PNA binding. Over time the number of R-beads build up on the specific sensors until the source target RNA is no longer available. The final(total) signal is then captured. The T-beads can be recycled 3911 after the elution step 3907 and returned back to the initial capture step 3903 as shown in FIG. 39. The T-beads may be cleaned before returning to the initial capture step 3903 to ensure the beads have no spurious probes, R-beads or RNA attached.

Where the CMOS detector is a single bead continuous flow sensor as described above, without sensor surface binding, assay building and elution follow the same with the T-bead recycled after elution. In such embodiments, the R-bead and T-bead are diverted post-elution with the R-bead flowing past the single bead CMOS detector and the T-bead returning to the sample at the initial capture step until the target RNA is depleted. The signal is then read at each event to effectively count the number of beads that have passed the CMOS detector.

### Single-Molecule Analysis

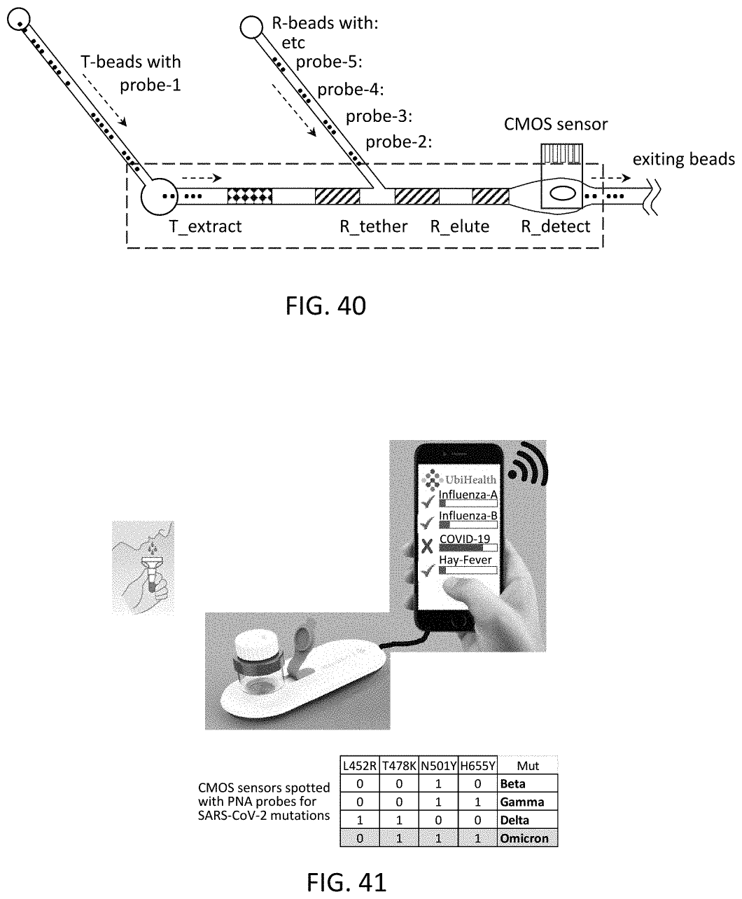

As discussed throughout, various signal amplification techniques can be used alone or in combination to increase sensitivity to, for example, allow for single-molecule detection. Single molecule detection has many applications including early detection of infections and in accurate quantification of viral load in, for example, SARS-CoV-2 outbreaks. FIG. 40 shows an embodiment for single-molecule analysis, e.g. of SARS-CoV-2.

In early 2022 Lai et al (DOI: 10.1101/2022.01.08.22268865) published a list of 48 markers or nucleotide mutations in the SARS-CoV-2 genome corresponding to the known variants at that time: Alpha, Beta, Gamma, Delta, Lambda, Mu, Epsilon, Iota, Eta, Kappa, and an early Omicron variant. Assuming a ‘brute-force’ set of 48 probes targeting each of these mutation areas in the genome, Probe-1 is a capture probe designed to uniquely target all SARS-COV-2 variants, as shown in FIG. 12 for example. Probe-1 may be attached to the assay's paramagnetic transport (T) beads, which flow continually to capture and extract the SARS-CoV-2 RNA from the sample. R-beads can also flow into a tethering chamber sequentially, in groups with different probes attached: firstly probe 2, then probe-3, then probe-4, etc, up to probe-48. The variant(s) in the patient sample can then be identified by a search and narrowing analysis, as partially illustrated in FIGS. 13 & 14. In some embodiments, the number of probes required can by reduced by applying a ‘truth-table’ logical reduction analysis to identify the variants, as shown in FIG. 41 with mapping of the L452R, T478K, N501Y, and H655Y mutations to identify Omicron variant.

FIG. 41 illustrates an assay embodiment for a home respiratory test from a self-test saliva sample. In addition to COVID-19 detection and variant identification of SARS-CoV-2 described above, extra probes are added to discriminate and identify Influenza-A, Influenza-B, RSV and other viral pathogens, and also peptide sequence probes of epitope antigen for detection of Immunoglobulin E (IgE) antibody, to identify Allergic Rhinitis (‘Hay-Fever’), which may be the cause of a bout of sneezing and congestion. The up to single molecule assay sensitivity, lack of enzymatic amplification (the might complicate at-home use), and the multiplex nature across classes of targets of the embodiments discussed above can all be combined to allow for such a test.

### Detection of Beads Flowing in Other Low Dielectric Constant Liquids

Other examples of low-dielectric-constant liquids are Dodecafluoro-2-methylpentan-3-one (Novec 649), and fluorinated carbons such as methoxy-nonafluorobutane (Novec 7100). These are clear, colourless and low odour liquids used as advanced heat transfer fluids with desirable environmental and electrical properties. Novec 649 is commonly used in electronics cooling. It has a very low dielectric constant (@1 kHz) of 1.8, making it an advantageous carrier medium for capacitance sensing of beads. Additionally, it is denser and poorly soluble with water, and as a fluorinated substance, it will fractionate away from both aqueous and oil-based liquids. In certain embodiments, beads released into a flow past a sensor may be monodispersed within that liquid to aid in assay sensitivity and single molecule detection. In some embodiments, a phase transition may be used as commonly the sample type will be aqueous.

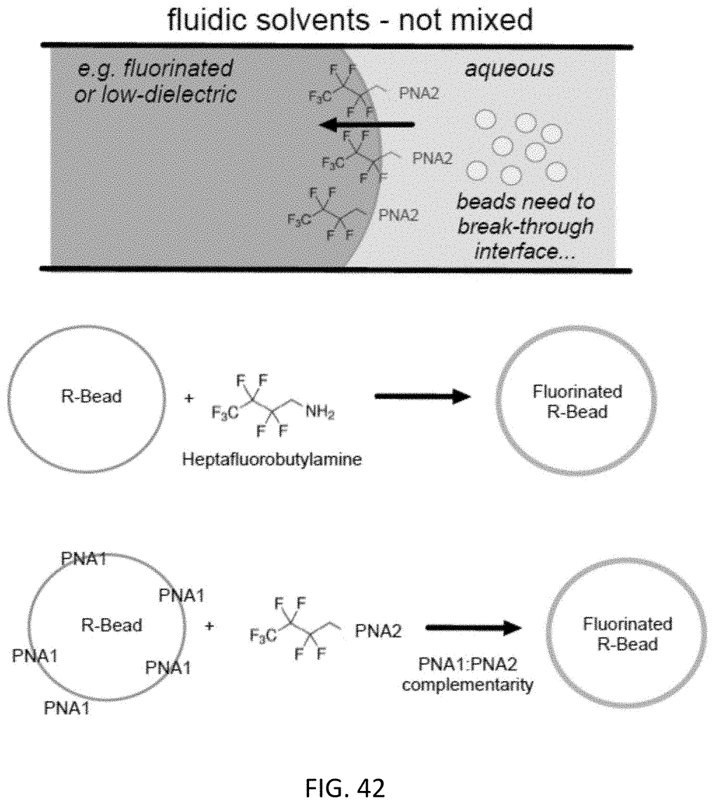

FIG. 42 shows schema for creating a fluorinated R bead at an interface between an aqueous and fluorinated solvent according to certain embodiments. The beads may be modified with a ligand that allows transfer across the interface. The interface is shown in FIG. 42 followed by beads being modified into fluorinated R-beads using, for example, heptafluorobutylamine (proven with Dynabeads) and beads modified with fluoro-PNA (e.g., Fluor-PNA2 will form micelles and/or align at an aqueous/fluorinated fluid interface). The scheme illustrates how a biphasic soluble PNA (PNA and fluorinated terminal) can be used to tag an aqueous dissolved R bead, allowing for subsequent transfer of the R bead to a fluorinated solvent. Similar chemistries are available for producing lipophilic beads for transfer to oil. In some embodiments, the R bead can have amphiphilic properties and be capable of being monodisperse in both aqueous and organic or fluorinated solvents. Additionally, the use of amphiphilic detergents dissolved in one or both solvents can facilitate transition of beads between solvent phases. This approach has been demonstrated by Wei et al (DOI: 10.1021/ja039874m, incorporated herein by reference in its entirety) for the transfer of gold nanoparticles from aqueous phase to an immiscible ionic liquid.

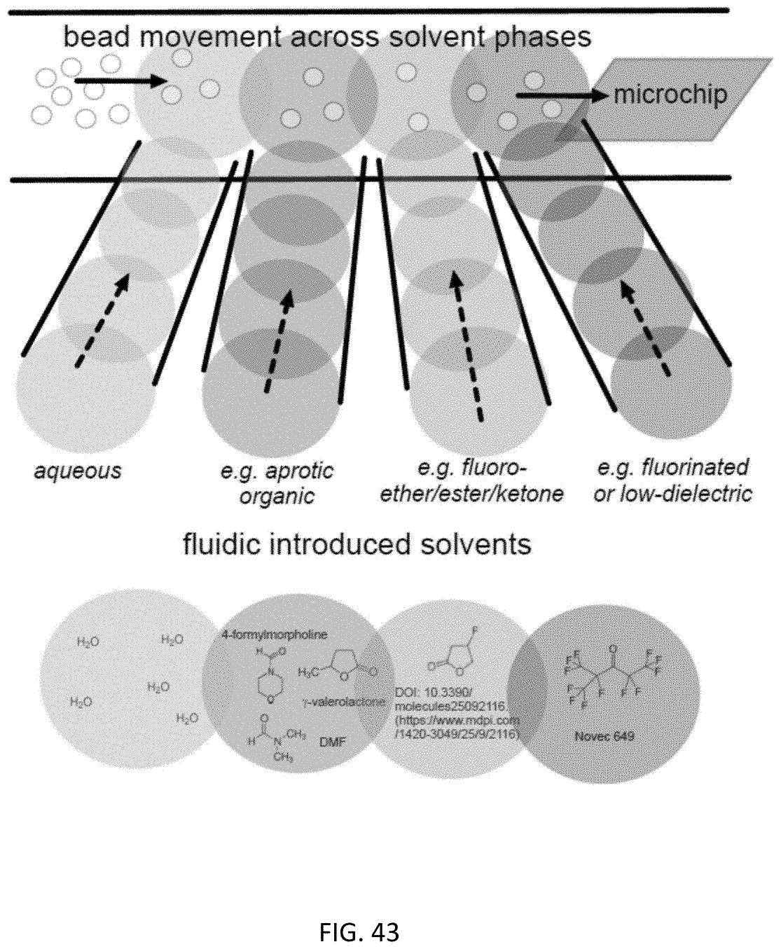

In certain embodiments, the transfer of beads to, for example, a final fluorinated solvent, may be by way of a transition between multiple solvents (e.g., aqueous to aprotic/organic, to fluoroesther/ester/ketones, to fluorinated or low-dielectric solvents). Such transfer can be performed using microfluidic approaches such as shown in FIG. 43. Fluidics can be used to introduce different solvents with overlapped mixing or micellar formation as the bead moves across the solvent phases. This approach may also facilitate the use of phase-transfer molecules or amphiphilic detergents in each of the separate solvents. This approach is guided by the Hansen-solubility parameters of the beads and the solvents, obtaining sufficient solubility of each to facilitate bead phase transfer. Similar methods may be used in bead production microfluidically, transferring beads from organic to aqueous solution.

Another way of moving the R beads from one solvent to another may be to exploit their high dielectric constant or a high Zeta potential to deflect the R beads from a flow of the “sample” or aqueous solution to a parallel flow of Novec 649 or other fluid wherein the beads move laterally across flow lines until they are in the Novec 649 and deflected to an outlet with the sensor.

In some embodiments, magnetically susceptible R-beads can be deployed in the device. As is shown in the literature (Pamme & Manz, 2004, DOI 10.1021/ac049183o, incorporated herein by reference in its entirety) magnetophoretic separation can be used to move beads through a fluid. Magnetophoretic separation may be used to deflect the R-beads from the aqueous phase to the Novec 649 or an oil-based or other phase.

The transfer of beads from an aqueous phase to an oil-based phase allows the formation of a thin shell layer or droplet of aqueous solution around the bead when the bead is in the oil based phase. In some embodiments, flowing this aqueous coated bead through a capacitance sensor can provide an amplified signal over that of the standard bead, due to the high dielectric constant of the aqueous shell layer on the bead, thereby further increasing system sensitivity

### Tethering and Bead-Flow Assay Combinations

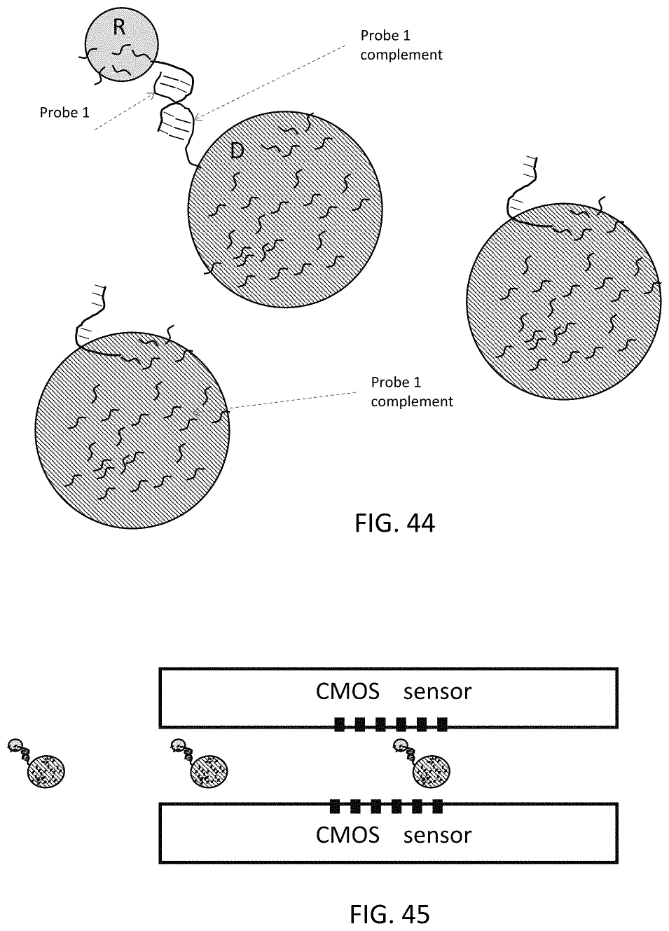

In FIG. 44 embodiment, R beads eluted from the upstream assay may subsequently become bound to a further bead to amplify the signal (‘D bead’, again mimicking dendritic amplification). This can be done using PNA-PNA binding between the beads. This allows the R bead to be smaller and allow more of these to be attached to a single molecule (e.g., RNA). FIG. 45 shows R-bead-D-bead complexes being measured going through a flow channel wherein two CMOS sensors are arranged atop each other to partially or entirely create the flow channel and the capacitance signal is measured by both chips independently as the beads move past each sensor. Measurements with one sensor are digitally compared to measurements from the other sensor. The same arrangement can be used for measuring single beads.

The R bead can be used to mediate capture of the R bead and D bead complex on a substrate. The complex may have properties which mediate differentiation from un-complexed beads. These may include capture of the complex on a substrate (mediated by the R bead PNA, for instance) which may complete the assay wherein the substrate is the surface of a sensor. The properties of the complex may include PNA-PNA binding, dielectrophoresis, or electrophoresis. The property may draw the complex to the sensor. The property may deviate the complex within a flow channel wherein the complex passes by the sensor and un-complexed particles flow through an outlet. The property may retain the complex or elements of the complex on the substrate temporarily while un-complexed particles are washed and/or removed via an outlet. For instance, where PNA-PNA binding to a substrate is used, the temperature of the system can be increased to elute one or other member of the complex from the substrate after un-complexed particles have been removed via an outlet whereafter it flows past a sensor and can be measured as described elsewhere herein. The capture and subsequent release of multiple beads can accomplish the aforementioned tethering signal amplification in flow-through embodiments.

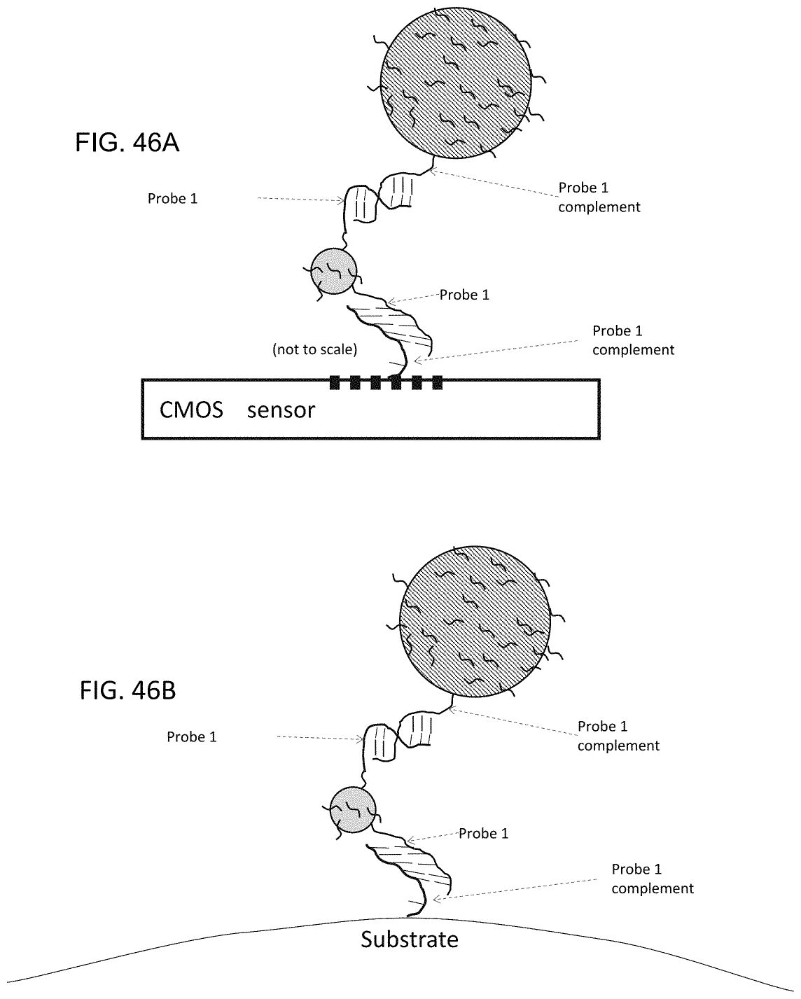

In certain embodiments, the R-bead may be reduced to a single PNA and binding of this PNA to another substrate can be used to mediate capture of the D bead. The substrate can be a sensor chip (i.e. functionalized with a PNA for PNA-PNA capture) as shown in FIG. 46A, or the substrate could be an additional magnetic bead as shown in FIG. 46B. This can provide for tethering and manipulation of beads magnetically with the advantage of the very strong PNA-PNA binding within each member of the tethered complex. Accordingly, the advantages in signal amplification through tethering can still be obtained when using a flow-through sensor.

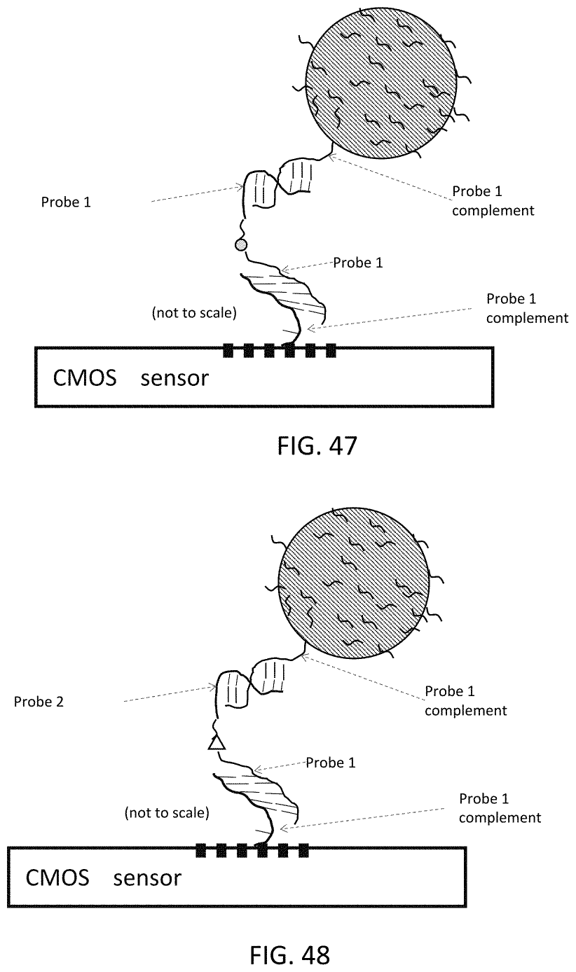

FIG. 47 is an example of a much smaller R-bead (1-100 nm) tethering D-bead to surface.

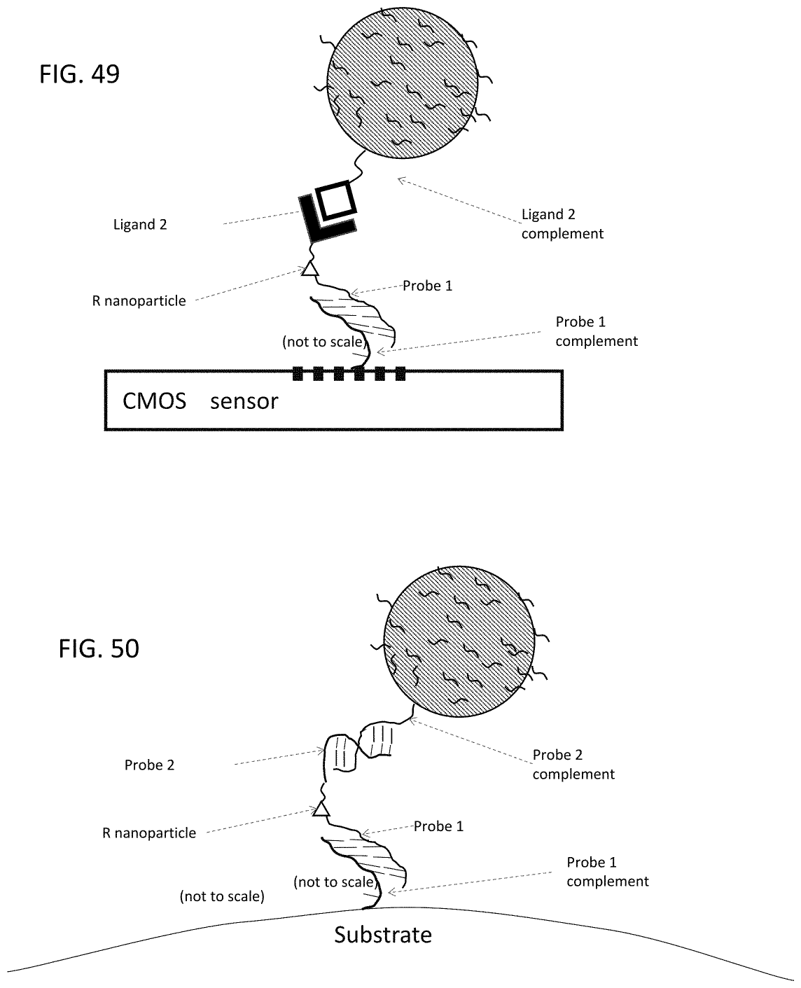

FIG. 48 illustrates that the R-nanoparticle can have other shapes, or be a linker-type molecule allowing the attachment of one or more probes, or be a long PNA probe with two regions. Probe 2 can be a probe or ligand which is also functionalized on the R nanoparticle but which does not interact with natural RNA target. This may be accomplished by careful probe design to avoid sequences found in the natural RNA target and to avoid interaction with the PNA probe. The ligand may be such that does not interact with normal base pairing on the target RNA including but not limited to biotin-streptavidin, as shown in FIG. 49.

FIG. 50 shows the reduced-size R nanoparticle (1-100 nm) tethering D-bead to surface of a magnetic particle for further manipulation ahead of sensing.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A method for nucleic acid detection, the method comprising detecting a target nucleic acid present in a sample at 100 copies/mL or less using a capacitive sensor and without enzymatic amplification.

2. The method of claim 1, operable to achieve at least 10⁹ signal amplification in detection of the target nucleic acid.

3. The method of claim 1, operable to achieve signal amplification in detection of the target nucleic acid equivalent to at least 30 PCR cycles.

4. The method of claim 1, wherein the nucleic acid detection by capacitive sensor comprises a full-scale range of at least about 8 pF, at least about 0.5 aF resolution, and at least about 4 aF accuracy.

5. The method of claim 4, further comprising converting a signal from the capacitive sensor using a sigma-delta 24-bit capacitive-to-digital converter capacitive-to-digital converter.

6. The method of claim 4, wherein the nucleic acid detection by capacitive sensor comprises a full-scale range of at least about 15 pF through inclusion of internal offset capacitors in a reference input.

7. The method of claim 1, further comprising:
binding the target nucleic acid to a reporter (R) bead;
passing the R-bead through a sensor region comprising two capacitive electrodes on a substrate in communication with a signal processing circuit,
wherein the two electrodes are spaced apart to form a gap, and
wherein only a single bead moves between the gap at a time; and
detecting the R-bead as it passes through the gap using the signal processing circuit.

8. The method of claim 7, further comprising:
passing the R-bead through a plurality of sensor regions; and
detecting the R-bead as it passes through the plurality of sensor regions by applying one or more of a Maximum Likelihood Estimation (MLE) machine-learning algorithm, a Partial-Response Maximum Likelihood (PRML) algorithm, or a Viterbi algorithm to signals received by the signal processing circuit from the plurality of sensor regions.

9. The method of claim 1, wherein detecting the target nucleic acid comprises:
binding the target nucleic acid to a reporter (R) bead; and
detecting the R-bead using a capacitive sensor.

10. The method of claim 9, wherein detecting the R-bead using the capacitive sensor comprises detecting the R-bead flowing past the sensor in a fluid.

11. The method of claim 9, further comprising binding the target nucleic acid to the R-bead using a bead-bound peptide nucleic acid (PNA).

12. The method of claim 1, further comprising detecting a plurality of different targets comprising at least the target nucleic acid present in a sample using one or more capacitive sensors.

13. The method of claim 12, wherein the plurality of different targets comprises a protein.

14. The method of claim 12, wherein detecting the plurality of different targets comprises:
binding each of the plurality of targets to a different bead; and
detecting each of the different beads using the capacitive sensor.

15. The method of claim 9, wherein detecting the target nucleic acid further comprises:
binding the target nucleic acid to a probe on a substrate; and
binding a plurality of reporter (R) beads to the substrate-bound target nucleic acid.

16. The method of claim 15, wherein the plurality of R beads bind to different sequences in the substrate-bound target nucleic acid.

17. The method of claim 16, wherein the substrate is operably associated with the capacitive sensor.

18. The method of claim 16, further comprising releasing the plurality of R-beads from the substrate-bound target nucleic acid;
flowing the released R-beads past the capacitive sensor in a fluid; and
detecting the released R-beads flowing past the sensor.

19. The method of claim 7, wherein the two electrodes form a trench through which the R-bead passes.

20. The method of claim 1, wherein detecting the target nucleic acid comprises:
binding a transport (T) bead to the target nucleic acid in the sample to form a T-bead complex;
binding the T-bead complex to a reporter (R) bead to form an R-bead complex;
eluting the R-bead and the T-bead from the R-bead complex;
detecting the R-bead using a capacitive sensor; and
returning the T-bead to the sample to bind another target nucleic acid.
